(19) United States
(12) Patent Application Publication
(10) Pub. No.: US 2016/0243502 A1
(43) Pub. Date: Aug. 25, 2016

(54) Volume Reduction Filtration Devices For Cell Suspension And Method

(71) Applicants: EXMOOR PHARMA CONCEPTS LTD, Reading Berkshire (GB); LONZA BIOLOGICS PLC, Slough (GB); UCL BUSINESS PLC, London (GB); AMERCARE LTD, Thame (GB)

(72) Inventors: John Vincent, Reading (GB); Rui Tostoes, London (GB); Farlan Veraitch, London (GB); John Dodgson, Slough (GB)

(21) Appl. No.: 15/033,263

(22) PCT Filed: Oct. 30, 2014
(86) PCT No.: PCT/GB2014/053228

§ 371 Date: Apr. 29, 2016

(30) Foreign Application Priority Data
Oct. 30, 2013 (GB) 1319141.6

(51) Int. Cl. B01D 63/08; C12N 5/071

(52) U.S. Cl. CPC B01D 63/08; C12N 5/0602; B01D 2313/10; B01D 2313/18; B01D 2313/32

(57) ABSTRACT

The present invention provides devices, apparatus and systems suitable for achieving volume reduction in a liquid containing a species in suspension and methods of use thereof. The apparatus of the invention comprises (i) a filter device comprising: a tubular first chamber having a filter provided in a surface thereof, a second chamber in fluid communication with the first chamber through the filter, the device being provided with an inlet port opening to the first end of the first chamber, an outlet port opening to the second end of the first chamber, and a filtrate port opening to the second chamber, and (ii) a fluidic pathway from the outlet port, wherein the apparatus is configured to introduce the first liquid and a second fluid immiscible with the first to the first chamber and the apparatus comprises a flow cut-off means in the fluidic pathway from the outlet port.

The present invention relates to devices, apparatus and systems suitable for achieving volume reduction in a liquid containing a species in suspension and methods of use thereof.

### BACKGROUND

Prior ad devices for filtration and diafiltration of a liquid containing a species in suspension are known to have drawbacks, especially where the aim of the filtration process is to recover the retentate. In the case of a dead-end filter the species accumulates on the fiber, restricting flow and resulting in reduced filtration efficiency, in the case that the object is to recover retentate, recovery rates are often low as the retentate adheres to or forms a cake on top of the filter. Tangential flow filtration (TFF, also known as cross-flow filtration), in which the suspension is flowed across a fitter such that a majority component of flow in the bulk liquid is parallel to the filter surface, reduces the tendency of the retentate to adhere to the filter and for cake to build up, and is especially suited to provide a continuous flow of filtrate from the filtration process it may be used to concentrate the retentate, but is essentially a multi-pass process in which a smelt proportion (typically less than 10%. often less than 5%) of the liquid in the suspension is removed at each pass through the and suffers the disadvantages ti) the species must be pumped around a recirculation pathway through the retentate side of the filter, which is undesirable if the species is easily damaged Of tends to adhere to the surfaces of the fluidic pathway: (ii) at higher concentration factors the retentate tends to become denser and more viscous and so harder to recirculate and, in some cases, more prone to damage to or agglomeration of the species and (iii) that TFF is usually implemented using a differential pressure across the membrane provided by a pressure drop component in the outlet flow pathway of the retentate side of the filter device, which for easily damaged species may add to the risk of damage, especially at higher concentrations.

Particularly, for the case of cell suspensions and processes in which the cells in the retentate are the valued output from the filtration or diafiltration process, such as in cell therapy bioprocessing, prior art devices and apparatus using them are poorly adapted, in particular, small scale TFF systems have a large internal surface area compared with the volume of the sample chamber and the area of the filter, formed by the surfaces of the recirculation system and the pump means, which leads to unacceptable losses of cells by adsorption to the surfaces.

In cell therapy cells having a therapeutic effect are administered to a patient, for example by topical injection or by systemic infusion. The cells may be sourced from the patient themselves in autologous therapy, cultured, selected or otherwise manipulated, sometimes expanded in number, and then transplanted back into the patient. In allogeneic therapies a donor cell line is cultured, cells are treated to add or to enhance efficacy, selected or otherwise manipulated, and then transplanted into a patient. In either case in many production processes the cells need to be cultured in a culture medium that is not suitable for administration to a patient, so which needs to be washed out and replaced by an excipient (a liquid medium usable as a carder for a therapeutic agent, suitable for use in the human body) before administration. In some cases the volume of the cell suspension needs to be reduced. In some cell therapy production processes a cell suspension may be frozen containing a cryoprotectant for storage and transportation, and needs to be thawed and the cryoprotectant washed out and replaced with an excipient before administration to the patient. Whether the cell suspension is used with freezing or without, there is a need for an apparatus adapted to carry out washing and volume reduction steps and then to enable loading 01 the product into an administration device ready for use. Such an apparatus needs to be able to process small volumes of valuable cells in suspension, achieving good recovery rates with acceptably low damage to the cells, preferably in a dosed system and in an automatic or semi-automatic way. The prior art does not provide apparatus and methods that meet this need.

It is an object of the invention to provide improved filter devices, improved apparatus using filter devices, systems comprising such devices and apparatus for processing suspensions, and methods for filtration of suspensions that overcome these and other drawbacks of the prior art it will be appreciated that while the devices, apparatus, system and methods will be described herein with reference primarily to cell suspensions, according to the embodiment they are usable for suspensions containing other species, such as particles and macromolecules.

### PRIOR ART

US2003024877, Amann et al., discloses a cell concentration device comprising a closed ended vertical tube having a filter in e wall of the tube and a flow pathway through the filter to a filtrate chamber, with optional vacuum aspiration via the filtrate chamber to assist flow. In use cell suspension is loaded into the tube and liquid from the suspension flows through the filter, so concentrating the suspension. The tube optionally has a tapered collection region below the fitter. Concentrated suspension may then be removed by a pipette or syringe. Also disclosed is a dosed tube having in the cap a septum through which the suspension may be loaded and recovered and a breather. This device allows cell concentration but does not allow for efficient washing of the cast no means is disclosed for efficient mixing of a wash liquid with the cells. The device is for manual operation and is not adapted to be built into automated or semi-automated equipment.

U.S. Pat. No. 8,394,631, Hampson et al., discloses a cell culture device comprising a fluidic channel having an inlet port and an outlet port, cells being cultured on a surface over which the channels pass. Cells on the surface are washed by passing wash liquid through the channels so as to cause plug flow to displace the culture medium without displacing the belts. No filter means is disposed in the fluidic pathway through the device. The volume of wash liquid within the device may then be reduced by introducing a gas so as to cause a plug flow within the channel and to displace the wash liquid out of the outlet port. This device has the disadvantage that in the absence of a fitter, cells are likely to be displaced along the channel and lost. The volume reduction step relies on cells being mobilised off the surface which contradicts the requirement for washing without cell loss. This device is adapted for culture of a high cell density and poorly adapted for washing a sample in which a high cell recovery percentage is desired.

WO97004857, Cui, discloses a filtration apparatus for suspensions using a gas uplift pumping mechanism, comprising a filter device having a vertically oriented filter separating a retentate chamber and a filtrate chamber, the retentate chamber having an inlet port at the base and an outlet port at the top, and a teed suspension reservoir connected to the mid port by means of a check valve and to the outlet port Gas under pressure is introduced by means of a T junction into the fluid line from the check valve to the inlet port. Gas/liquid mixture in the sample chamber rises, forcing recirculation of the feed suspension from and to the reservoir through the filter device. The feed reservoir is under positive pressure, so forcing filtrate through the filter where it is recovered. The feed suspension is concentrated and may be recovered from the reservoir. This apparatus is again poorly adapted for filtration and volume reduction of a small sample; no collection device is provided to allow complete recovery of the concentrate from the reservoir and no fluidic connection suitable for this purpose is disclosed.

Cheng T W et al. in ‘Effects of gas slugs and inclination angle on the ultrafiltration flux in tubular membrane module’. J. Membrane Sci. 1999, 158(1-2): p 223-234, disclose a tangential flow filtration (TFF) device operated at an angle between vertical and horizontal, having a retentate chamber and a filtrate chamber separated by a tubular filter, an inlet port at the base of the retentate chamber and an outlet port at the top, and a fluidic circuit connected to the device comprising a feed tank connected to the inlet port Via a pump, a source of compressed air coupled by a T-junction to the flow line from the pump and thence to the inlet port, and a return line to the feed tank connected to the outlet port via a flow control valve operable to provide a pressure drop in the return line in order to create a pressure difference across the filter to drive filtrate through the fitter, as known in the art of TFF. This apparatus is again poorly adapted for filtration and volume reduction of a small sample; no collection device is provided to allow complete recovery of the concentrate from the reservoir and no fluidic connection suitable for this purpose is disclosed, in the case where the suspension is a cell suspension, the flow control valve in a partially open state to provide a pressure drop will impose a shear stress on the cells, potentially damaging them.

In summary, prior art filtration apparatus of the types described above is generally better adapted for separation of a liquid filtrate from a species such as cells or other particles in suspension, when the frigate is the desired fraction, than when the retentate cells or particles are the desired fraction. In particular, the prior art devices are poorly adapted for concentration of a species in a small volume of retentate and for the efficient unloading of that small volume from the filter device into a collection device. Where such prior art devices are usable, they are manual devices requiring a complex series of operations by a skilled user and so are not adapted for larger scale or automated use. The prior art apparatus described above oath suffer from one or more of the following drawbacks: (i) they operate on a large volume scale and are impractical for operation at reduced size; (ii) owing to pressure drop within the system or passing of concentrate through a lengthy or recirculating fluidic pathway they are likely to damage a shear-sensitive retentate particle such as a cell or to suffer losses of cells to walls of the apparatus; (iii) they do not include an effective means for unloading a small volume of concentrated suspension from the titter device; (iv) they do not offer a volume reduction capability within the device; (v) they do not include a filter means to retain the species, so rely on slow liquid flow rates and adherent or settled particles (such as cells) in low concentration, hence leading to long process times and potential unreliability, and (vi) they are inherently manually operated or open system devices and as such are not capable of integration into an automated or closed-system apparatus.

In particular, the prior art does not address the need for efficient filtration and volume reduction of small volumes of suspension were the retentate particles are the desired fraction, and where the particles may be precious and where high recovery is essential. Specifically in cell bioprocessing where the cells are the desired fraction, as for example in cell production and processing for cell therapy, the prior art apparatus is unsuitable.

Definitions

Herein unless otherwise stated a liquid means an aqueous liquid that may comprise solutes, such as water, a salt solution, and acidic or basic solution, a buffer, a physiological liquid such as blood or a blood fraction, seminal fluid saliva or urine, a liquid medium as used in cell culture, cell differentiation, cell processing, transport or cryopreservation, or a liquid medium as used in chemical synthesis, analysis or separation, or in materials processing including minerals extraction. The liquid may comprise a mixture of liquid compounds, such as a mixture of water and miscible manic compounds. Where stated the liquid may be a non-aqueous liquid, comprising one or more liquid components that are non-miscible with water, such as a oil, short chain paraffin or other organic compound in liquid form, which may comprise solutes such as other organic compounds or inorganic compounds soluble in the organic compound,

A suspension is a liquid containing a species in suspension. Herein a suspension is an aqueous suspension unless otherwise stated. The phrase ‘in suspension’ means contained within the liquid and in some cases being hydrated, having surface-associated components from the liquid, having a surface charge resulting from interaction with the liquid, or having a liquid environment around it altered from a bulk environment in the same liquid where the species is not present. In the case that the species is a macromolecule such as a protein, the term suspension herein includes a liquid containing a solubilised macromolecule.

An undesired component may be a solute or a compound mixed with the liquid, a fraction of the liquid or a species in suspension in the liquid alongside a species of interest. For example, an undesired component may be a constituent of a culture medium in a cell suspension, a component of a cryoprotectant solution added to a cell suspension before freezing, such as dimethyl sulphoxide (DMSO), a salt or a stabilisation component in the case that the species is a protein such as an antibody or a contaminant of the suspension such as a contaminating solid fraction, protein, endotoxin or virus.

In separating a first species from a second, the first species may comprise particles or other species of a first size or type and the second species particles of a second size or type, for example a first species may be a white blood cell and a second type may be a red blood cell, a first species may be a specific type of cell and a second species may be another type. A first species may be a cell and a second species a non-cell particle. A first species may be an intact cell and a second species may be lysed cell fragments.

‘Single pass filtration’ herein means that the suspension to be filtered is passed through a tiller device once. In contrast, TFF as disclosed in the prior art is a multi-pass filtration operation in which the suspension to be filtered is recirculated through the filter device and may pass through it many times during the filtration or volume reduction process.

A species may be one or more of a cell, a vesicle, a liposome, a virus, a micro-organism, a bacterium, a particle, for example a solid particle or a semi-liquid particle such as a droplet in an immiscible liquid, for example an aqueous droplet in a non-aqueous liquid or vice versa, a particle formed from a gelled polymer, a nanoscale or microscale particle such as a shell structure, a core-shell particle, a nanotube or a nanorod, an assembly of cells such as is coli cluster or an embryoid body, a microcarrier or other substrate, one or more cells on such a microcarrier or one or more cells bound together by a natural substrate such as a protein or a complex comprising multiple proteins, or a macromolecule such as a nucleic acid, an antibody or another protein. A species may comprise two or more other species bound together, for example a cell bound to one or more particles, or two or more particles bound together, for example by a protein-protein or nucleic acid-nucleic acid interaction.

Herein nanoscale means having a lamest characteristic dimension of the order of 500 nm or smaller, for example in the range about 500 nm to 5 nm or in the range 100 nm to 10 nm, and in preferred embodiments in the ranges about 500 nm to 50 nm, 100 nm to 10 nm, or 50 nm or smaller. Microscale means having a largest characteristic dimension in the range of the order of 500 um to 0.1 μm or in the range about 100 μm to 0.5 μm and for example in preferred embodiments in the ranges about 100 μm to 10 μm, about 50 μn to 5 μm about 10 μm to 1 μm, about 5 μm to 0.5 μm, about 1 μm to 0.1 μm. A shell structure is a substantially hollow particle having a shell that is either closed or nearly so. A core-shell particle is a particle having a core and a shell coating the core, formed from a different material from the core. A nanotube (for example a carbon or boron nitride nanotube) is a hollow elongated nanoscale particle, and a nanorod (for example a gold nanorod) is a solid elongated nanoscale particle, each having a ratio of its largest to its smallest dimension of typically 5 or greater. A microcarrier is a particle adapted for culture of cells adherent to the surface of the particle. Typically microcarriers are microscale particles, for example 125-250 micrometre spheres though they may have other shapes such as flat plates, for example hexagonal in shape (Nunc Microhex). Microcarriers can be made from for example DEAE-dextran (Cytodex, GE Healthcare), glass, polystyrene (SoloHill Engineering), acrylamide, and collagen (Cultispher, Percell). Microcarriers may be porous or non-porous and have a specific surface, coating, such as collagen, or surface chemistry, including for example extracellular matrix proteins, recombinant proteins, peptides, and positively or negatively charged molecules.

A particle may be a cell, virus, bacteriophage, plasmid, chromosome, or polymer.

A cell may be a prokaryote cell or a eukaryote cell, or a collection of cells in the form of a tissue or tissue fragment, or other multicellular body, for example an embryoid body. A cell may be for example of the following types: a eukaryote cell such as a plant cell, a plant spore, or an animal cell such as a mammalian cell.

A mammalian cell may be a human cell, for example as listed below:

a keratinizing epithelial cell, such as a keratinocyte, a hair shaft cell, a hair root sheath cell, a hair matrix cell (stem cell), a cell of the wet stratified barrier epithelia such as a surface epithelial cell;

a cell specialized for secretion of hormones such as beta cells of the pancreas, cells of the pituitary gland, or a cell of the gut or respiratory tract;

a cell specialized for metabolism and storage such as a hepatocyte, a liver lipocyte or a fat cell;

an epithelial cell serving primarily as barrier function, lining the tang, gut, exocrine glands, or urogenital tract, an epithelial cell lining closed internal body cavities such as vascular endothelial cells of blood vessels and lymphatics, a synovial cell, a aerosol cell, a squamous cell of the ear, a choroid plexus cell (secreting cerebrospinal fluid);

a cell from the following list squamous cells of the pia-arachnoid, cells of the ciliary epithelium of the eye, corneal “endothelial” cells, a ciliated cell with propulsive function of the respiratory tract, of the oviduct and of the endometrium of the uterus (in a female), of rote testis and ductulus efferens (in a male) or a cell of the central nervous system, a cell specialized for secretion of extracellular matrix, of non-epithelial tissue (connective tissue) such as fibroblasts for example of loose connective tissue, of the cornea, of the tendon, of the reticular tissue of bone marrow, a pericyte of blood capillary, a nucleus pulposus cell of the intervertebral disc, a cementoblast/cementocyte (secreting bonelike cementum of the root of the tooth), an odontoblast/odontocyte (secreting dentine of the tooth), chondrocytes of hyaline cartilage of fibrocartilage of elastic cartilage, an osteoblast/osteocyte, an osteoclast, an osteoprogenitor cell (stem cell of osteoblasts), a hyalocyte of the vitreous body of the eye or stellate cell of the perilymphatic space of the ear;

a contractile cell such as skeletal muscle cells, heart muscle cells, smooth muscle cells, or myoepithelial cells of the iris or of the exocrine glands;

a cell related to blood or the immune system such as a red blood cell, megakaryocyte, macrophages and related Gets (monocyte, connective-tissue macrophage, a Langerhans cell (in epidermis), an osteoclast On bone), a dendritic cell (in lymphoid tissues), a microglial cell (in the central nervous system)), a neutrophil, eosinophil, basophil, mast cell, T lymphocyte (helper T cell, suppressor T cell, killer T cell), B lymphocyte (IgM, IgG, IgA, IgE), or killer cell, or stem cells and committed progenitors for the blood and the immune system;

a cell with sensory and transducing functions such as photoreceptors (rod, cones), hearing, acceleration and gravity, taste, smell, blood pH, touch, temperature, pain and configurations of, and forces in, the musculoskeletal system;

a cell from the following list: an autonomic neuronal cell, a supporting cell of the sense organs and of peripheral neurons such as supporting cells of the organ of Corti, or a supporting cell of the vestibular apparatus, or a supporting cell of the taste bud, or a supporting cell of the olfactory epithelium, or a Schwann cell, ore satellite cell or an enteric glial cell; a neuronal or glial cell of the central nervous system such as neurons or glial cells (astrocytes, oligodendrocytes);

a cell from the following list: a lens cell, a pigment cell such as a melanocyte or a retinal pigmented epithelial cell, a germ cell such as an oogonium/oocyte, a spermatocyte, or a spermatogonium, a nurse cell such as ovarian follicle cell, a Sertoli cell (in the testis), or a thymus epithelial cell, an interstitial cell, or an interstitial cell of the kidney or other organs with pacemaker functions;

a cell from the following list: embryonic stem cells, induced pluripotent stem cells, mesenchymal stem cells, mesenchymal stromal cells, very small embryonic-like stem cells, neuroblasts, myoblasts, hepatoblasts, pancreatic progenitor cells, cardiac progenitor cells, hematopoietic stem cells, retinal progenitor cells, photoreceptor precursor cells, corneal progenitor cells, keratinocyte progenitor cells, adipose stem cells, dental pulp stem cells and all adult cell types originated from embryonic stem cells, induced pluripotent stem cells, very small embryonic-like stem cells and adult stem cells.

A vessel is a container having an interior volume adapted to hold a fluid. A vessel may be substantially rigid or may comprise a flexible well in the manner of a polymer bag adapted to contain a liquid. A vessel may be formed for example as a void in a solid body; as a void formed by two recesses in the major surface of two components held together to define a substantially closed volume; as a volume defined within a set of solid walls that may have a thickness that differs in different areas of the wall—for example the wail thickness may be small compared with a characteristic dimension of the vessel. A vessel may be formed for example by injection moulding of one or more three dimensional polymer components; by machining, embossing or moulding a number of components that are then bonded or otherwise held together; by extruding a profile, optionally followed by processing the extrusion substantially to close one or both ends. The invention is not limited to as specific size or volumetric capacity of vessel but in certain embodiments is adapted for use with a small vessel, suitable for a small volume of suspension. A vessel herein may have a volume in the range 0.05 ml to 5 l, in the range 0.5 ml to 500 ml, in the range 5 ml to 50 ml, and in preferred embodiments in the range 0.5 ml to 5 ml, in the range 1 ml to 10 ml, in the range 5 ml to 50 ml, in the range 10 ml to 100 ml, in the range 50 ml to 500 ml, in the range 100 ml to 1 l or in the range 500 ml to 5 l. In some embodiments suitable for large volume filtration the vessel may have a volume greater than 5 l.

A filter is an element for separation of a species from a first liquid and from small molecule components dissolved the first liquid. In some embodiments the filter may allow a first species to pass through it while preventing a second species from passing. The filter may compose a filter membrane, which in some embodiments is hydrophilic. Herein the material of the filter is chosen to allow a first liquid born the suspension and where present a wash liquid miscible with the first liquid to pass through it while preventing the second phase from passing through it once the filter has been contacted by the first liquid. In the case that the first liquid is aqueous and the second phase is a gas, the fitter is preferably a hydrophilic membrane having a bubble breakthrough pressure selected to be greater than the maximum pressure applied across the fitter in use, such that gas wilt not break through the filter, in the case that the second phase is a second liquid immiscible with the first the filter is chosen such that it is not wetted by the second liquid given prior wetting by the first liquid. According to the embodiment, filter membranes may comprise a material chosen from the non-exclusive group ok hydrophilic membrane mater regenerated cellulose, Polyvinyl difluoride (PVDF), cellulose acetate, mixed cellulose ester, polyethersulfone, polysulphone, or polycarbonate in the case of track-etched membranes such as Nuclepore™ or Cyclopore™ filter membranes (Whatman, UK). In some embodiments the filter membrane may be formed from a hydrophobic filter membrane treated to render it hydrophilic, for example by washing in surfactant. Fitters having pore sizes in the range of 0.1 μm to 10 μm are available from a number of suppliers and the devices and methods of the invention are usable with a range of filter types. In some embodiments the filter may comprise a mesh such as a nylon or stainless steel mesh, for example having an opening size in the range 30 to 100 μm as used for filtration of larger particles and cell clusters, agglomerations, sheets or multicell bodies. Filter types used for separating cells from liquid or for removing contaminating microorganisms from cell suspensions typically have a pore size in the range around 0.1 to around 1 μm.

A planar filter is a substantially planar fitter component that may be in the form of a substantially flat sheet, or may have deviations from being flat such as corrugations, bowing in one or two dimensions, ripples or dimples. A planar fitter comprises a filter curved in one or two orthogonal dimensions, for example a domed filter, having a perimeter substantially in a single plane. The term ‘plane of the filter’ means a plane characterising a planar filter when in situ in a filter device, for example the plane of the majority of the perimeter of the filter or a plane forming the mean position of a corrugated, rippled, dimpled or bowed filter.

An immiscible fluid herein means a fluid that forms a separate phase when in contact with the liquid of the suspension, for example a gas in contact with a liquid, a non-aqueous liquid in contact with an aqueous liquid, and a second aqueous liquid in a two-phase aqueous system as formed for example by solubilised macromolecules provided in one or both phases. In some cases an immiscible liquid may form a mixed phase with the liquid of the suspension. According to the embodiment the filter may be chosen according to the nature of the immiscible fluid to allow the liquid of the suspension to pass through it but not, or to a lesser extent, an immiscible second fluid.

Herein the first chamber refers to the chamber into which the suspension is flowed in use, and corresponds to the retardate chamber in literature descriptions of conventional Tangential Flow Filtration (TFF) devices. The first chamber is not limited to a specific size or volume of but in certain embodiments the invention is adapted for use with a small first chamber volume, for example approximately 10 ml or less, suitable for a small volume of suspension. A first chamber herein may have, a volume in the range about 0.05 ml to about 5 l, in the range 0.5 ml to 500 ml, in the range 5 ml to 50 ml, and in preferred embodiments in the range 0.5 ml to 5 ml, in the range 1 ml to 10 ml, in the range 5 ml to 50 ml in the range 10 ml to 100 ml, in the range 50 ml to 500 ml, in the range 100 ml to 1 l or in the range 500 ml to 5 l. In some embodiments suitable for large volume filtration the first chamber may hay a volume greater than 5 l, in some embodiments the first chamber may have a first cross-sectional dimension parallel to the filter in the range about 5 mm to 500 mm, in the range 10 mm to 200 mm, or in the range 20 mm to 100 mm, and in preferred embodiments in the range 5 mm to 50 mm, in the range 10 mm to 100 mm, in the range 50 mm to 500 mm. The first chamber may have a value of the second cross sectional dimension perpendicular to the filter divided by the first cross-sectional dimension parallel to the filter in the range about 0.01 to 2, 0.02 to 0.5, and 0.05 to 0.1.

Herein the second chamber refers to the chamber into which filtrate liquid passes from the first chamber via the filter and corresponds to the filtrate chamber in literature descriptions of conventional TFF devices. The second chamber is not limited to a specific size or volume, and in certain embodiments may have a similar volume, to that of the first chamber. In some embodiments the second chamber may have a volume smaller than that of the first chamber, for example serving primarily to collect liquid passing through the filter from the first, chamber so that the liquid may be directed to a filtrate reservoir. In some embodiments the second chamber may have a greater volume that the hist chamber, for example in order to function as a filtrate reservoir. A second chamber herein may have a volume in the range about 0.05 ml to about 5 l, in the range 0.5 ml to 500 ml, or in the range 5 ml to 50 ml.

A vertical or a horizontal orientation defines the orientation as shown in the figures and as indicated by vertical and horizontal axes.

A port is defined as an opening to a vessel through which a fluid and a species may pass.

A fluidic pathway means a pathway along which a fluid may flow, such as may be formed for example within tubing or fluidic components such as valves, pumps, manifold and fluidic connection devices, and may be defined at least in part within a solid body component, for example m the form of channels, apertures, junctions, or interfaces between a first body component and a second, and by passages through a porous material such as a fitter material, a porous membrane or a venting component such as a hydrophobic porous material adapted to allow gas to pass Put not to wet with aqueous liquid, for example porous fluoropolymer (such as VYON™, Porex, UK). A fluidic pathway may compose a valve means operable to control flow along the pathway, for example to open or to close the fluidic pathway to fluid flow.

The phrase ‘tapers towards a port’ means that at least one cross-sectional dimension of a chamber or vessel decreases as the port is approached. For example, referring to the first chamber, in some embodiments the first chamber tapers in one cross-sectional dimension, in some embodiments the first chamber tapers in two cross-sectional dimensions. Such a taper may comprise a cross sectional shape that remains unchanged, or a shape that changes, as the port is approached. For example the first chamber may have a first shape being one of a rectilinear, triangular, oval, circular, semi-oval or semicircular cross-sectional shape that changes to a second, different shape the above group as the port is approached, in an embodiment the first chamber tapers from a rectilinear to a circular shape. In some embodiments the port comprises a perimeter in the surface of the first chamber and the cross-sectional shape of the first chamber changes from a first shape at the plane of the filter to a circular cross-sectional shape, in a direction perpendicular to the axis of the fluidic pathway leading through the port. According to the embodiment a cross-sectional dimension may decrease continuously or discontinuously, for example step-wise. For example the first chamber may comprise a rapid change of cross-sectional dimension at a step or ledge, or at a point of change of shape for example from a square to a circular cross-section. According to the embodiment a taper provides an area of a planar cross-section of the first port at its opening to the first chamber of less than 50%, 50% to 20%, 20% to 5%, less than 5%, or less than 1% of the area of the filter.

A first feature or component being ‘in fluid communication’ with a second means that a fluid may flow from the first to the second or vice versa,

A first feature or component being ‘connected to’ a second herein moans the first is ‘in fluid communication with’ or ‘optionally in fluid communication with’ the second. A first component may be .connected to a second via a valiire. When the valve is open a fluidic pathway is provided between the first component and the second and when the valve is closed the two are still ‘connected’, i.e. the option of thud communication is present, but the two are not in fluidic communication and the fluidic pathway no longer exists between them.

‘Directly connected’ herein means that a first component is connected to a second without an intervening component—for example a collection device is directly connected to a filter device if there are no further components such as a valve in the fluidic pathway between them.

A valve means is any means to control flow along a fluidic pathway. A valve means may be a pinch valve or a valve comprising an internal fluidic; pathway, and according to the embodiment may be controllable to out off or to limit flow proportionally according the embodiment. A 3-way or 4-way valve means may comprise a plurality of discrete 2-way valves manifolded together or may comprise a multi-position changeover valve. A valve means may be provided as part of a filter device in some embodiments, in other embodiments a valve means may comprise a discrete valve component mounted on, adjacent to or separately from the fitter device as part of the apparatus, in some embodiments a valve means may be a cheek valve. A valve means may also compose a fluidic actuation means that controls flow along a fluidic pathway. A valve means may comprise a syringe where the plunger is held in place so preventing fluid flow into the syringe, for example held in place by an actuator. A valve means may comprise a pump where flow is limited or prevented when the pump is not actuated.

A valve means may comprise a valve actuator and a flexible tubing portion, wherein the valve actuator is adapted to receive the tubing portion so allowing it to close the fluidic pathway through the tubing portion, so forming a pinch valve, in some embodiments the tubing portion is separable from the valve actuator such that it may be mounted onto the valve actuator and removed from it.

A flow cut-oft means is a means to prevent flow along a fluidic pathway. A flow cut-off means has two stable conditions in a first ‘closed’ condition flow is prevented along the fluidic pathway leading to or through it and in a second ‘open’ condition flow is allowed. A flow cut-off means may be a out-oft valve that has a first open position and a second closed position. A flow cut-off means may also be a device such as a syringe or pump that when stationary prevents fluid flow along a fluidic pathway into the device. A flow cut-off means may comprise means to create backpressure in the fluidic, pathway to prevent fluid flow along it, such as a venting pathway comprising a valve, such that when the valve is closed the fluidic pathway does not vent, so causing backpressure preventing a fluid entering the fluid pathway. In some embodiments a flow cut-off means may be a septum such that the fluidic pathway is opened when the septum is pierced. In some embodiments the flow cut-off means may form part of the collection device. In some embodiments the collection device may be a syringe having a plunger whose movement is controlled by an actuator such that while the plunger is held stationary the syringe itself acts as a flow cut-off means to prevent flow along the fluidic pathway into the syringe. In some embodiments the collection device may comprise a pump connected to a container such that when the pump is not actuated the pump acts as a flow cut-off means.

A cutoff valve has a closed condition in which flow along the fluidic pathway through it is prevented and an open condition in which the valve causes substantially no pressure drop along the fluidic pathway at the flow rates intended for use in the apparatus. The invention is not limited to any specific flow rate, and the cut-off valve may he chosen by the skilled person so as not to present a pressure drop through it when open as compared with pressure drops in other parts of the apparatus. In some embodiments the cut-off valve may comprise a pinch valve, a 2-way valve having wetted internal components, or a 3-way or higher-way valve having the capability to close the fluidic) pathway between a first port and a second port on the valve.

A check valve is a valve that allows flow in a first direction but net in the reverse direction, and is pressure sensitive: a positive pressure in the direction of allowed flow opens the valve, and a negative pressure in that direction (i.e., a positive pressure in the reverse direction) causes the valve to close.

A source of a fluid may comprise a reservoir containing the fluid such as a rigid reservoir, a collapsible reservoir or bag (a flexible fluid container formed by sealing together of flexible polymer layers), or a syringe, and in some instances may be actuatable in order to control flow of fluid into or out from the reservoir, for example by compressing a bag or applying a head pressure of gas to a liquid within a reservoir. A source of fluid may comprise a gaseous atmosphere external to the apparatus, or a source of fluid from a reservoir connected to the apparatus but remote from it.

A reservoir configured to retain a fluid allows a fluid to flow into it in a first flow direction but does not permit exit of the fluid in the same flow direction. For example, such a reservoir for a liquid may comprise a container having a fluidic inlet and a breather, so allowing liquid to enter the reservoir and gas to leave it, liquid being retained within the reservoir. In some embodiments liquid may exit the reservoir once again if the flow direction is reversed. Such a reservoir may comprise a bag, which inflates as it fills with fluid.

A fluidic actuation means is a device for moving fluid along a fluidic pathway, for example a pump such as a positive displacement pump, a peristaltic pump, a syringe, a source of liquid at a height to provide flow under gravity, pressure in a liquid reservoir such as gas pressure in the headspace above a liquid in a reservoir, and compression of a compressible fluidic reservoir.

A collection device is a device adapted to collect the output of a process carried out in the apparatus, for example a concentrated suspension, and may comprise a passive collection device such as a receptacle or an active collection device comprising a receptacle and a fluidic actuation means. A collection device may comprise one of the following: a container, a vial, a catheter, a tubeset adapted to receive the product before administration to a patient, a flexible bag, a syringe, a syringe with a pre-fitted needle, a pump having a fluidic pathway to a receptacle.

A breather is a device in fluid communication with a reservoir or a fluidic pathway adapted to allow gas to pass through it but to prevent passage of liquid out from the reservoir or pathway. For example a breather for use with aqueous liquids may comprise a hydrophobic porous material through which gas may pass but which will not be wetted by an aqueous liquid, or a narrow hydrophobic capillary channel into which an aqueous liquid will not pass. For example a breather may comprise a porous fluoropolymer such as VYON™, Porex, UK. A breather for use with non-aqueous liquid may comprise a hydrophilic material that will not be wetted by the non-aqueous liquid.

Washing and diafiltration are taken to have the same meaning herein, that a component of a liquid suspension is reduced in concentration or replaced by one or more components from a second liquid added to the suspension.

A wash liquid refers to a liquid miscible with the liquid of the suspension, adapted to wash a component out from the suspension. Herein the wash liquid and the liquid of suspension are aqueous unless otherwise stated. A wash liquid may be for example water, saline, a buffer, an aqueous solution containing solutes such as salts or sugars for example to control osmolarity, an acid, base or neutral solution. A wash liquid may comprise a complexing agent to complex a component of the suspension. For example, in some embodiments the suspension is a cell suspension comprising a cryoprotectant arid the wash liquid is adapted to replace at least a portion of the cryoprotectant. In some embodiments the wash liquid may he an excipient suitable for injection into a patient. In some embodiments the wash liquid has an osmolarity similar to that of the cell suspension to avoid osmotic shock.

A cryoprotectant is a material that when added to a cell suspension allows the suspension to be frozen while allowing the cells to recover on thawing. Examples of cryoprotectants are dimethyl sulphoxide (DMSO) and solutions containing it, typically at between 5 and 10% by volume though in some cases a higher concentration; glycerol, trehalose, sucrose and polyethylene glycol (PEG) in a range of concentrations, and proprietary cryostorage solutions such as Cryostore™ (Biolife, Inc. USA).

An excipient is a liquid medium usable as a carrier for a therapeutic agent, suitable for use in the human body. Examples of excipients include phosphate buffered saline (PBS) or physiological saline solution.

A culture medium is a liquid in which cells may be cultured containing nutrients, buffers, salts and proteins required for cell growth. The composition of the medium may vary depending en the cell type to be cultured.

A dosed system is an apparatus whose interior is isolated from the external environment, and within which process steps may be carded Out without exposing an interior portion or surface of the apparatus, or its contents or materials that contact the contents during the process, to the external environment so that following sterilisation of the apparatus It may be used in aseptic processing without needing to be within a clean environment.

### DESCRIPTION OF THE INVENTION

The filter device, apparatus, system and method of the invention is directed to filtration, including diafiltration, of a liquid containing a species in suspension, which is referred to also as a ‘suspension’, for example concentration of the species, washing out an undesired component from the liquid or from within the species, separation of the species from the liquid, or separating a first species within the liquid from a second. In particular the apparatus is adapted for single pass filtration of a suspension.

In a particular application and in some embodiments the spades are cells and the invention is directed to one or more of separation, filtration, diafiltration, concentration and resuspension of cells. In particular in some embodiments the device apparatus and method are adapted for processing cells in suspension prior to use in cell therapy.

According to a first aspect of the invention there is provided an apparatus for filtration of a first liquid comprising a species in suspension, comprising:

(i) a filter device comprising a tubular first chamber having a filter provided in a surface thereof arid a second chamber in fluid communication with the first chamber through the filter, the device being provided with an inlet port opening to the first end of the first chamber, an outlet port opening to the second end of the first chamber, and a filtrate port opening to the second chamber, and

(ii) a fluidic pathway from the outlet port,

wherein the apparatus is configured to introduce the first liquid and a second fluid immiscible with the first to the first chamber and the apparatus comprises a flow cut-off means in the fluidic pathway from the outlet port.

In some embodiments the inlet port is configured to receive both the first liquid and the second immiscible fluid.

In some embodiments the how cut-off means comprises a cut-off valve connected to the outlet port.

In some embodiments the flow cut-off means comprises means to prevent fluid flow into a collection device.

In some embodiments the flow cut-off means comprises a septum.

In some embodiments the apparatus comprises a collection device directly connected to the outlet port. In some embodiments the collection device comprises a syringe.

In some embodiments the apparatus comprises a control means configured to set the flew cut-off means in one of a first condition in which flow along the outlet fluidic pathway is prevented and a second condition in which such flow is permitted.

In some embodiments the apparatus comprises a control means configured to set a cut-off valve in one of a first position in which the valve is closed and a second position in which the valve is open. In some embodiments the control means comprises one or more power supply switches to control the power supply to one or more of cut-off valves, pumps and actuators in some embodiments one or more such switches are manually-operated switches. A control means may comprise a computer controlled electronic system that interprets instructions in the farm of data and provides an output to control one or more valves, pumps or actuators configured to control movement of fluids into and out from the fitter device. The valves, pumps or actuators may be configured to control movement of fluids in one or more fluidic components or pathways connected to the device, for example in a fluidic unit as described herein. The control means may compose a computer such as a microprocessor, a data storage means readable by the computer and a user interface. The control means may comprise communication means to communicate data to or from a display or a further electronic system or data store. The apparatus further comprises a power supply to power the control means and optionally to power valves, pumps or actuators controlled by the control means. The control means may be configured to receive data entered by a user, received from a remote electronic system or data source, read from a data storage means associated with the filter device such as with a fluidic unit as described herein, and to control movement of fluids in response to such data. The control means may be configured to output data to a display, a printer, a remote electronic system, computer or data storage means, such as a data storage means associated with a fluidic unit comprising the filter device.

In some embodiments the cut-off valve in the open position has substantially no pressure drop along the flow pathway through the valve at the now rates intended for use in the apparatus. The invention is not limited to any specific flow rate, and the cut-off valve may be chosen by the skilled person so as not to present a pressure drop through it when open as compared with pressure drops in other parts of the apparatus. In some embodiments the cut-off valve itself has two conditions, namely fully open and fully dosed, and the control means is configured to move the valve between those two conditions.

In some embodiments the flow cut-off means forms part of the collection device. In some embodiments the flow cut-off means comprises a pump means adapted to prevent flow though it while not in motion, in some embodiments the flow out-off means composes a syringe. In some embodiments the flow cut-off means comprises a pump. In some embodiments the apparatus comprises a control means configured to control a syringe by means of a syringe driver or configured to control the pump. In this way when the syringe or pump are stationary flow along the outlet fluidic pathway is prevented and the control means enables flow by actuating the syringe or pump. In some embodiments the collection device in the form of a syringe, or of a pump plus a receptacle, provides a flow cut-off means in the outlet fluidic pathway controllable by the control means between a first condition in which the syringe or pump are held stationary and flow along the outlet fluidic pathway is prevented and a second condition in which the syringe or pump is actuated and said flow is permitted.

In some embodiments a cut-off valve may comprise a pinch valve, a 2-way valve having welted internal components, or a 3-way or higher-way valve.

In some embodiments the control means is configured to control flow into the first chamber of one or both of the first liquid and the second fluid.

In this way the apparatus provides a means for filtration and volume reduction of a suspension when introduced unto the first chamber and a second immiscible fluid is also introduced into the first chamber, as described in the detailed description herein.

In some embodiments the control means is configured to control flow out from the second chamber.

In some embodiments the apparatus further comprises a suspension inlet fluidic pathway comprising a suspension inlet valve and a second fluid inlet fluidic pathway comprising a second fluid inlet valve, the said fluidic pathways being connected to the first chamber, wherein the control means is configured to control the flow cut-off means and one or both of the suspension inlet valve and the second fluid inlet valve.

In some embodiments the control means is configured to set the said valves in a first condition in which the flow cut-off means allows flow out from the outlet port, the suspension inlet valve is open and the second fluid inlet valve is closed and in a second condition in which the flow cut-off means prevents flow out from the outlet port, the suspension inlet valve is closed and the second fluid inlet valve is open.

In some embodiments the suspension inlet valve is a check valve. In some embodiments the second fluid inlet valve is a check valve.

In some embodiments the apparatus comprises a source of a second fluid immiscible with the first liquid in fluid communication with a port opening to the first chamber.

In some embodiments the second immiscible fluid is a gas. For example in some embodiments the gas is an inert gas, nitrogen, air, artificial air, CO₂ in air or a CO₂/N₂/O₂ mixture for example to maintain pH in liquids containing a bicarbonate buffer. The gas may be humidified.

In some embodiments the second immiscible fluid is a second liquid. For example the second fluid rosy be a non-aqueous liquid in the case that the first liquid is aqueous.

In some embodiments the second fluid is an aqueous fluid immiscible with the aqueous liquid of the suspension, for example containing a solubilised macromolecular component that renders it immiscible with the liquid of the suspension. In some embodiments the suspension also comprises a solubilised macromolecular component that acts to render the liquid of the suspension immiscible with the second fluid.

In some embodiments the source of the second fluid comprises a reservoir containing the second fluid. A reservoir may be a container or may comprise a fluidic actuation means, for example a pump means. A reservoir may comprise a syringe, a rigid container, a flexible container, a supply of a fluid remote from the apparatus and connected to it by a fluid flow line.

In some embodiments the filter device comprises a cutoff valve. In some embodiments the filter device may comprise a housing having a first and a second chamber formed within it, the housing comprising a mounting location for a cut-off valve and the filter device provides a fluidic pathway from the outlet port of the first chamber to the cut-off valve, for example as formed by one or both of channels defined within the housing and tubing connected to the valve.

In some embodiments the cut-off valve is a check valve, configured such that flow is allowed out of the outlet port in response to a higher pressure at the outlet port than in the outlet fluidic pathway downstream of the check valve.

In some embodiments the flow cut-off means is in close connection to the first chamber as defined herein. ‘In close connection’ means that the outlet dead volume is less than the volume of the first chamber, where the outlet dead volume is the volume in the fluidic pathway between the outlet port of the first chamber and the flow cut-off means, in some embodiments the outlet dead volume is between about 0.005 and about 1.0 times the volume of the first chamber, for example in the range about 0.1 and 10 times the volume of the first chamber, in some embodiments between 0.02 and 0.2 times, in some embodiments between 0.005 and 0.05 times and in some embodiments less than about 0.005 times the volume of the first chamber.

The apparatus of the invention benefits from such a close connection as suspension that occupies the volume between the filter and the flow cut-off means is not in contact with the filter and so is likely not to undergo volume reduction by introduction of immiscible fluid. Efficiency of volume reduction is therefore increased if that volume is reduced.

Embodiments of the invention are configured to provide a volume reduction ratio, that is the ratio of the volume of suspension introduced to the first chamber to the volume of concentrated suspension after the volume reduction process, in the range about 1 to about 20, in some embodiments in the range about 2 to about 10, and in some embodiments in the range about 3 to about 6.

In some embodiments the filter is provided only in a portion of the wail of the first chamber. In some embodiments the first chamber comprises a region having impermeable walls downstream of the filter area through which liquid may pass. Such a region is referred to as a ‘concentration region’. Referring to the ‘active volume’ of the first chamber as the volume within which the filter surface intersects a line perpendicular to the centroid of the fluid flow pathway through the first chamber, in some embodiments the outlet dead volume is less than or approximately equal to the active volume, in some embodiments between 0.1 and 1.0 times the active volume, in some embodiments between 0.02 and 0.2 times the active volume, in some embodiments between 0.005 and 0.05 times and in some embodiments less than 0.005 times the active volume.

In some embodiments the apparatus further comprises a reservoir of a third liquid miscible with the first liquid in fluid communication with the inlet or the outlet port. Such a third liquid may be a wash liquid miscible with the hist liquid and adapted to replace a component within the first liquid. A wash liquid may be a culture medium, a buffer, a reagent, a stabilising agent, an agent to control or alter osmolarity, or an excipient. In particular in applications where the suspension is a cell suspension, the first liquid may comprise a cryoprotectant and the wash liquid may be adapted to replace the cryoprotectant. Such a wash liquid may be for example culture medium, a buffer, saline or an excipient.

In some embodiments the first chamber has a circular cross section and the filter is disposed in the surface forming the periphery of the first chamber.

In some embodiments the fitter device comprises one or more substantially planar membranes. Examples of suitable planar membrane filters are given in the preceding definitions.

In some embodiments the first chamber tapers towards one or both of the inlet port and the outlet port.

In some embodiments the filter device comprises a plurality of first chambers. Two or more first chambers may be in fluid communication with a common second chamber. Such first chambers may be filled in parallel for example from an inlet manifold, volume reduction may be carried out in the plurality of first chambers, and then concentrated suspension may be unloaded from the first chambers either in series or in parallel.

In some embodiments the filter device composes a housing having a filter permanently bonded to the housing. In some embodiments the filter device comprises a filter that is removable from the vessel. In some embodiments the filter device is adapted to be disassembled to allow the filter to be replaced. In some embodiments the filter is provided in a holder adapted to interfit with the housing to bin the vessel and the first and the second chambers.

In some embodiments the first chamber comprises a first wall impermeable to the first liquid and the filter is disposed in the surface of the first chamber distally to the impermeable first wall. For example such a device may be formed as a substantially planar structure having a planar fitter disposed above an impermeable base.

In some embodiments the device is configured with the filter substantially above all or the majority of the first chamber.

In sonic embodiments the outlet port is provided at substantially the lowest point of the first chamber.

In some embodiments a wall of the first chamber is inclined downwards towards the outlet port at an angle to the horizontal. In some embodiments the angle is in the range approximately 3 to approximately 85 degrees, and in some embodiments the angle is in the range approximately 30 to approximately 80 degrees.

In some embodiments the first chamber is arranged such that the filter is substantially vertical. In some embodiments the first chamber is arranged such that the filter is at an angle to the horizontal, for example between 5 and 45 degrees from horizontal, between 35 and 70 degrees or between 60 and 90 degrees from horizontal.

In some embodiments the device comprises one or more further ports opening to the first chamber at one or more positions between the inlet port and the outlet port. For example in some embodiments a plurality of inlet fluidic pathways for the second immiscible fluid are provided at intervals along the length of the first chamber. In this way during the volume reduction step a number of slugs of the second fluid may be introduced into the first chamber.

In some embodiments the filter device is in fluid communication with a collection device. According to the embodiment the apparatus is adapted to flew concentrated suspension out from the first chamber by means of pressure applied to an inlet port or by means of fluidic actuation associated with the collection device as defined previously.

In some embodiments the filter device and apparatus comprising it are adapted to form a closed system for processing a suspension. Such embodiments may be adapted for aseptic processing, for example for processing a cell suspension in bioprocessing, such as in processing a cell suspension for cell therapy.

Accordingly, in some embodiments the invention provides a fluidic unit composing a fitter device as described herein and one or more fluidic, pathways provided between the fitter device and components of the fluidic unit. The fluidic unit may comprise a reservoir connected to the filter device.

In some embodiments the invention provides an apparatus comprising a fluidic unit and a processor, together configured to carry out a process within the fluidic unit, the fluidic unit comprising the filter device and being adapted to interfit with the processor, the processor comprising one or more actuators configured to control fluid flow within the fluidic unit and a control means configured to control the actuators. In some embodiments the actuators are valve actuators.

In some embodiments the fluidic unit comprises:

a filter device as described herein,

an inlet adapted to receive a suspension,

a port opening to a first chamber of the fitter device,

a fluidic pathway connecting the inlet to the port,

a fluidic pathway configured to introduce a second liquid to the first chamber, and

an outlet fluidic pathway leading from a pod opening to the first chamber.

In some embodiments the fluidic unit comprises a collection device detachably connected to the outlet fluidic pathway.

In some embodiments the fluidic unit further comprises one or more of: a reservoir containing the second liquid connected to the first chamber of the filter device; a waste reservoir connected to the second chamber; a reservoir of gas connected to the first chamber.

In some embodiments the fluidic unit is configured to provide a closed system within the fitter device and other fluidic components and the fluidic pathways connecting them. In some embodiments the fluidic unit and/or fluidic pathways and components within it are sterilised such that the fluidic unit is adapted for use in aseptic processing, for example of a cell suspension.

In some embodiments the fluidic unit comprises a flow cutoff means connected to a port opening to the first chamber.

In some embodiments the flow cut-off means comprises a syringe forming part of the fluidic unit. In some embodiments the flow out-off means comprises a septum. In some embodiments the flow cut-off means comprises a cut-off valve comprising a fluidic pathway within the fluidic unit and an external valve actuator configured to close the said fluidic pathway.

In this way in some embodiments the fitter device and apparatus are adapted to achieve processing of a cell suspension, for example for use in cell therapy, in which a component is at least partially removed from the cell suspension by the washing process, and may be replaced by the wash liquid. The wash liquid may be an excipient, and thereby the suspension is processed ready for administration to a patient.

The fluidic unit may comprise one or more reservoirs in the form of a bag, vial or a syringe, and one or more connections between the filter device and a reservoir may be formed using tubing, for example flexible tubing, as known in the art.

The fluidic unit may compose additional components as described herein, for example one or more of the following: further inlets, for example for a second liquid such as a wash liquid and a fluid immiscible with the suspension for use in volume reduction; one or more sensors such as a flow sensor or a pressure sensor; one or more breathers adapted to allow passage of gas into or out from a fluidic pathway or reservoir while preventing the entry of pathogens into the fluidic pathway or reservoir. The fluidic unit may comprise one or more reservoirs containing fluids, for example a reservoir containing a wash liquid or a reservoir containing an immiscible fluid for use in volume reduction, such as e gas.

The fluidic unit may comprise a tube set comprising a filter device connected to one or more reservoirs in the form of a bag or a syringe by means of flexible tubing as known in the art.

The fluidic unit may comprise a tube set as defined herein and a housing adapted to contain the tube set and to retain the components of the tube set in a chosen configuration, in this way the fluidic unit may be mourned on the processor such that the tube set is located by the housing such that the processor may control fluid flow within the fluidic unit. It will be understood that the housing may retain the tube set while allowing a range of movement of one or more components or regions of the tube set so as to allow an actuator provided on the processor to come into contact with a portion of the tube set.

The processor may comprise one or more actuators such as valve actuators, configured to come into contact with and to move a region of the fluidic unit, for example to move a syringe plunger, to compress a bag, to compress a region of flexible tubing to move fluid within the tubing or to close the fluidic pathway through the tubing.

The apparatus may comprise one or more sensors for properties of a fluid, such as for flow, pressure, presence of liquid or gas such as bubble sensors), a chemical sensor such as a pH sensor or dissolved oxygen sensor, a conductivity or turbidity sensor; a level sensor to detect the level of a liquid in a reservoir; a temperature sensor, for example to measure temperature of a fluid within a fluidic pathway, of a component of the fluidic unit such as the filter device, or of a region within the fluidic unit; a proximity sensor or an orientation sensor, as may be used for example to detect correct mounting of the unit on a processor, or connection of a fluidic component such as a collection device to the fluidic unit. Such sensors may comprise a reading portion forming part of the processor and a fluidic sensor portion forming part of the fluidic unit and comprising a fluidic pathway, together forming a sensor, the reading unit providing data to a control means forming part of the processor.

The fluidic unit may comprise a data storage means that may be read by a data reader provided as part of the processor. Such a data storage means may comprise one or mom of a bar code, a 2D bar-code, a printed label, a magnetic data storage means such as a magnetic strip, an electronic data storage means such as a memory chip, a passive (read only) RFID chip or an active (read/write) memory chip. The processor may comprise means to read a data storage means associated with the fluidic unit.

The fluidic unit may comprise one or more electronic components such as sensors, indicators, switches or data storage devices configured to interact with one or more electronic components provided as part of the processor, and electrical contact means to make contact with contact means provided on the processor.

In some embodiments the apparatus comprises temperature control means to control the temperature of the filter device and optionally of further fluidic components connected to it. In some embodiments comprising a fluidic unit as described herein the apparatus comprises temperature control means to control the temperature of part or substantially the whole of the fluidic unit. A temperature control means may comprise a heater, a temperature sensor and a temperature control device adapted to control the heater in response to data from the temperature sensor. In some embodiments the temperature sensor is positioned to measure the temperature of a region of the filter device. In some embodiments comprising a fluidic unit and a processor comprising a control means, the control means is configured to receive temperature data from the temperature sensor and to control the operation of the heater. In this way the apparatus is configured to achieve processing of a suspension, for example a cell suspension, at a controlled temperature.

According to a further aspect, the invention provides a system for processing a liquid containing a spades in suspension comprising apparatus as described herein and a source of liquid containing the species in fluid communication with the inlet port of the filter device.

In some embodiments the system further composes a data source in data communication with the control means operable to control movement of liquid containing the spades within the apparatus. In some embodiments the data source comprises a computer and a database external to the apparatus. In some embodiments the data source comprises a data store physically associated with a container housing the liquid containing the species.

According to a further aspect the invention provides a method tar filtration of a first liquid containing a species in suspension using an apparatus or system as described herein, comprising the steps of:

(i) causing a first liquid containing the species to flow into the first chamber

(ii) introducing a second immiscible fluid into the first chamber while the fluidic pathway from the outlet port is closed, while liquid flows through the filter to the second chamber, to form a concentrated suspension containing the species in the first liquid in the first chamber,

and subsequently

(iii) flowing the concentrated liquid containing the species in suspension formed in step (ii) out from the first chamber.

In some embodiments the method comprises a step wherein the second fluid is introduced into the first chamber under pressure to force the first liquid through the filter.

In some embodiments that second fluid is a gas.

In some embodiments the second fluid is introduced into the first chamber until the volume of first liquid within the first chamber is reduced by between approximately 50% and approximately 98%. In some embodiments the second fluid is introduced into the first chamber until the volume of first liquid within the first chamber is reduced by between approximately 60% and approximately 95%.

In some embodiments the method comprises the further step of separating at least a portion of the second fluid from the first liquid.

In some embodiments the method comprises the further step of introducing into the first chamber a third liquid miscible with the first liquid, for example a wash liquid.

In some embodiments the first liquid is a cell suspension comprising a cryoprotectant.

According to as further aspect the invention comprises a suspension obtained by a method as described herein.

Preferred features of the second and subsequent aspects of the invention are as for the first aspect mutatis mutandis

### BRIEF DESCRIPTION OF THE FIGURES

FIG. 1a shows a first embodiment of an apparatus according to the invention.

FIG. 1b shows the embodiment in FIG. 1a in the process of volume reduction.

FIG. 1c shows the embodiment in FIG. 1b later in the process of volume reduction.

FIG. 1d shows the embodiment in FIG. 1c when volume reduction is complete end the concentrate is being moved out of the device.

FIG. 1e shows a further embodiment in the process of volume reduction.

FIG. 1f shows an embodiment of an apparatus according to the invention comprising a control means.

FIG. 1g shows an example of a process controlled by a control means as shown in FIG. 1f.

FIG. 1h shows a further embodiment of an apparatus according to the invention.

FIG. 2a shows a plan view of a further embodiment of en apparatus according to the invention.

FIG. 2b shows a cross-section view of the embodiment in FIG. 2a.

FIG. 3 shows a further embodiment of an apparatus according to the invention.

FIG. 4a shows a further embodiment of an apparatus according to the invention.

FIG. 4b shows a further embodiment of an apparatus according to the invention.

FIG. 5 shows a further embodiment of an apparatus according to the invention.

FIG. 6 shows a further embodiment of an apparatus according to the invention.

FIG. 7 shows an further embodiment of an apparatus according to the invention.

FIG. 8 shows an embodiment of an apparatus as shown m FIG. 1 following sequential volume reduction operations.

FIG. 9 shows a further embodiment of an apparatus according to the invention.

FIG. 10 shows a plan view of a further embodiment of an apparatus according to the invention.

FIG. 11 shows a cross sectional view of the embodiment shown in FIG. 10.

FIG. 12 shows the embodiment in FIG. 10 in the process of volume reduction.

FIG. 13 shows a cross-section through an embodiment of a filter device as shown in FIG. 1a.

FIG. 14 shows a cross-section through a further embodiment of a filter device as shown in FIG. 1a.

FIG. 15 shows an isometric view of a first variant of an embodiment of a filter device forming part of an apparatus of the invention, with characterising dimensional parameters of the device.

FIG. 16 shows an isometric view of a second variant of an embodiment of a filter device forming part of an apparatus of the invention, with characterising dimensional parameters of the device.

FIG. 17 shows a further embodiment of an apparatus according to the invention comprising a fluidic unit adapted for use with a processor configured to control movement of fluids within the fluidic unit.

### DESCRIPTION OF EMBODIMENTS

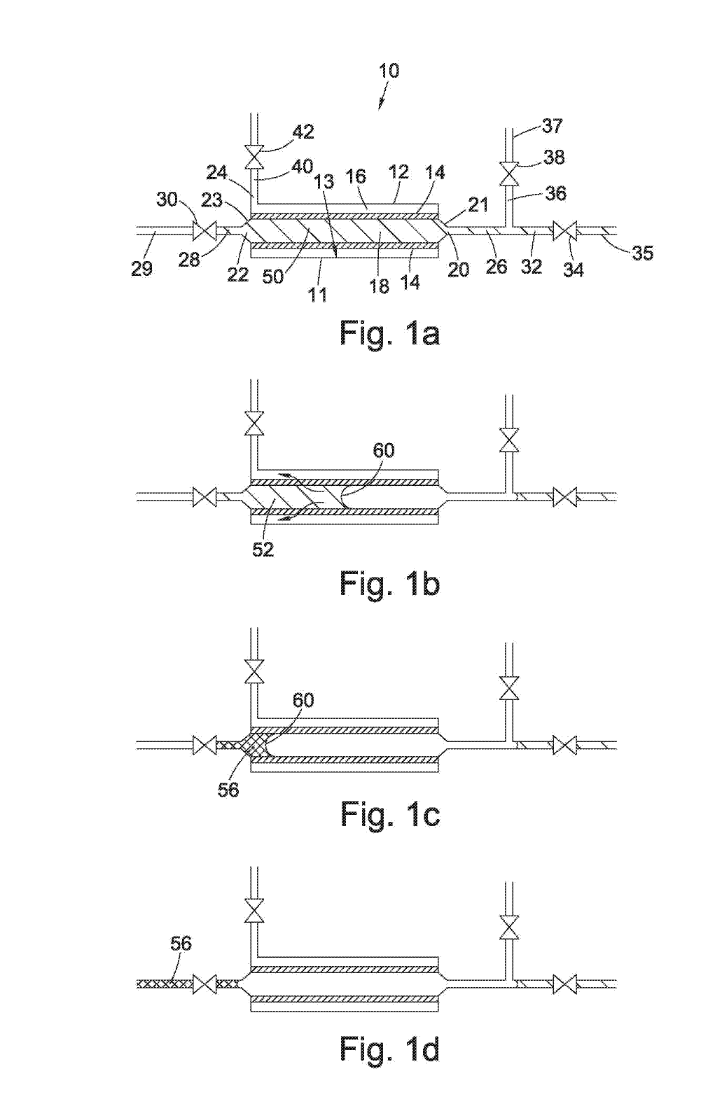

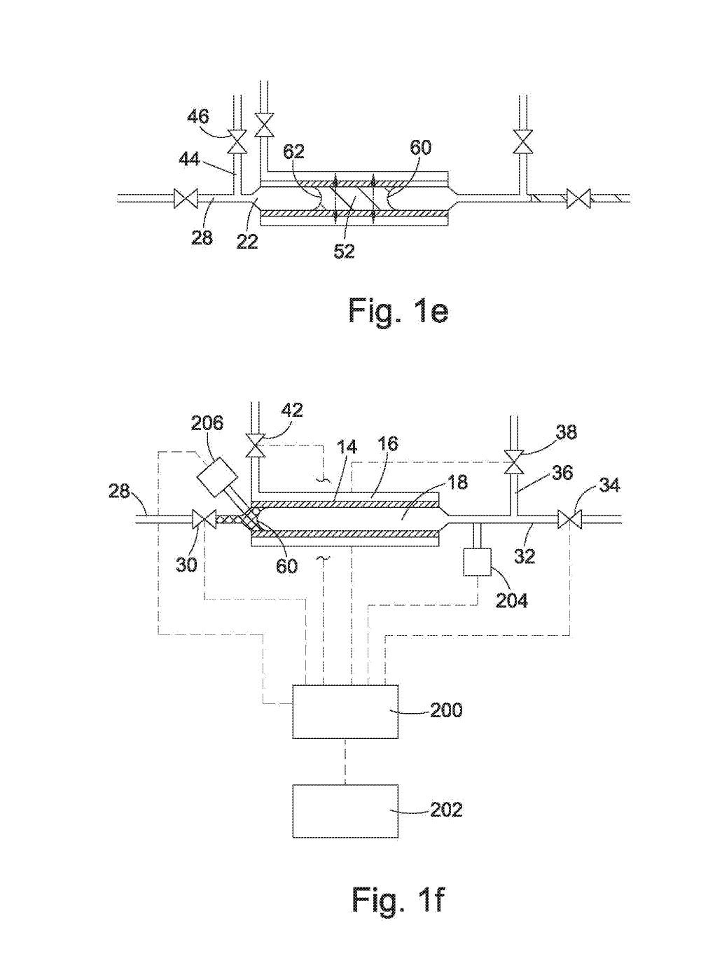

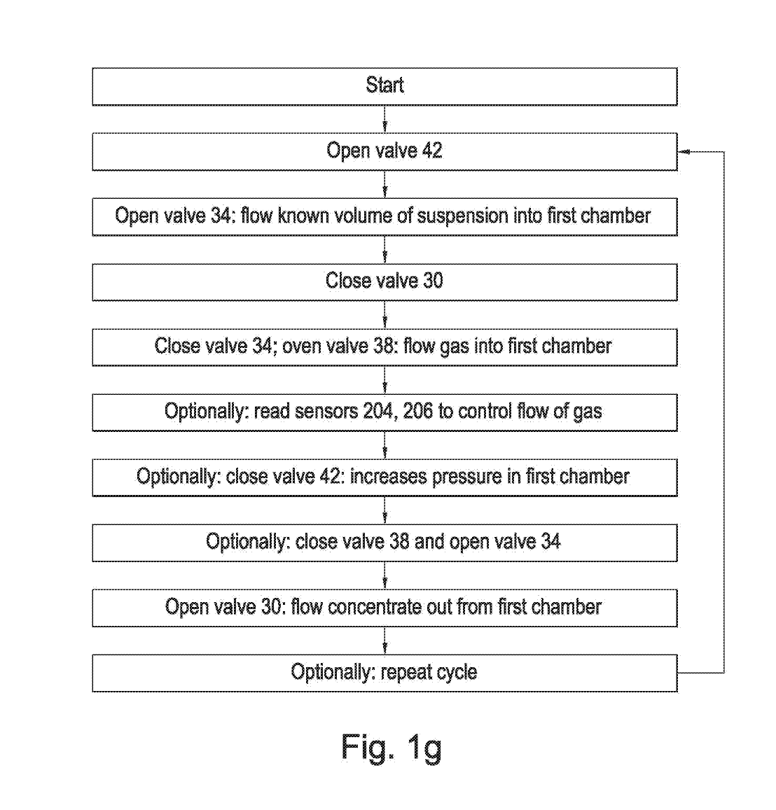

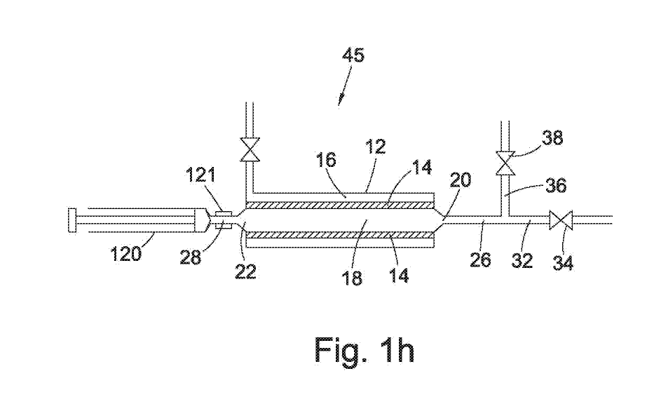

Referring to FIGS. 1a to 1d, an apparatus 10 for filtration of a first liquid comprising a species in suspension comprises:

(i) a filter device 12 comprising: a tubular first chamber 18 having a filter 14 provided in a surface thereof, a second chamber 16 in fluid communication with the first chamber through the filter, the device 12 being provided with an inlet port 20 opening to the first end of the first chamber, an outlet port 22 opening to the second end of the first chamber, and a filtrate 24 pod opening to the second chamber, and

(ii) a fluidic pathway 28 from the outlet port 22,

wherein the apparatus is configured to introduce the first liquid and a second fluid immiscible with the first to the first chamber and the apparatus comprises a tow cut-off means in the form of a cut-off valve 30 in the fluidic pathway 28 from the outlet port.

The inlet port 20 is further configured to receive both the first liquid and the second immiscible fluid.

The apparatus comprises an inlet fluidic pathway 26 connected to the inlet port 20 and to a suspension inlet fluidic pathway 32 and a second fluid inlet flow pathway 36. The apparatus further composes a suspension flow control valve 34 in the fluidic pathway 32 and a second fluid control flow valve 38 in the fluidic pathway 38. Optionally the apparatus comprises a filtrate flow control valve 42 in the titrate fluidic pathway 40 connected to the filtrate pod 24.

The cut-off valve 30 is adapted to have two positions: fully closed, and open, preferably having a substantially zero or at most a small pressure drop across the valve in the open position. In some embodiments the cut-off valve 30 is a remotely controlled valve and comprises a valve actuator having an electrical connection to a control means.

In some embodiments the cut-off valve 30 is a check valve having the characteristic of allowing flow in the sense from port 22 outward along the fluidic pathway 28 but not in the reverse sense.

In the apparatus of the invention cut-off valve 30 differs from flow control valves as used in the prior art in the technique of tangential flow filtration (TFF) in which a pressure drop is imposed along an outlet fluidic pathway from a filter device in order to create a pressure differential across a filter. Here life requirement is for a valve that has two stable conditions: in a first condition the valve stops the flow along the outlet fluidic pathway 28 and in a second condition the valve allows flow with little or no pressure drop across it.

The cut-off valve may comprise a valve as known in the art, such as a pinch valve.

A method of filtration of a first liquid containing a species in suspension using the apparatus of figures la id comprises the following steps:

(i) causing a first liquid containing the species to flow into the first chamber 18

(ii) introducing a second immiscible fluid into the first chamber while preventing flow out from the first chamber along the fluidic pathway 28 from the outlet port 22, while liquid flows through the filter 14 to the second chamber 16, to form a concentrated suspension containing the spades in the first chamber,

and subsequently

(iii) flowing the concentrated liquid containing the spades in suspension formed in step 50 out from the first chamber.

In some embodiments the method comprises the further steps of (iv): controlling the cut-off valve 30 such that it is closed before step (ii), and (v): opening the cut-off valve 30 before step (iii) above.

As shown in FIG. 1a, a suspension 50 is introduced into the first chamber by means of inlet fluidic pathways 32, 26 under control of valve 34. In some embodiments cut-off valve 30 is dosed while the suspension is introduced. In some embodiments cut-off valve 30 is open while the suspension is introduced. The volume of suspension introduced into the first chamber may be controlled by means of the valve 34, by controlling a fluidic actuation means operating to cause suspension to flow along inlet fluidic pathway 32, or both. In some embodiments the volume of suspension introduced into the first chamber may be greater than the volume of the first chamber, in which case liquid from the suspension will pass through the filter to second chamber 10. As shown in FIG. 1b, a second fluid immiscible with the first liquid is then introduced into the first chamber via fluidic pathway 36 and inlet fluidic pathway 26 under control of valve 38, while cot-off valve 30 is closed. As the second fluid is unable to pass through the filter 14 once the filter has been contacted by the first liquid, the meniscus 80 advances through the first chamber while liquid from the suspension passes through the filter 14, forming a more concentrated suspension 52. Closed cut-off valve 30 prevents exit of the suspension along the outlet fluidic pathway 28. As shown in FIG. 1c as the meniscus moves further along the first chamber volume reduction proceeds leaving a still more concentrated suspension 58. The volume reduction process may be controlled by valve 38 in terms of the amount of second fluid introduced into the first chamber by valve 38. As the meniscus nears or reaches the end of the first chamber and the liquid Bow pathway from the concentrated suspension to the filter becomes more limited, the pressure drop across the filter wilt tend to increase and, in the case where a constant pressure is applied to the gas, movement of the meniscus tends to slow arid may stop. A control means may monitor the pressure in the first chamber or in the fluidic pathway connected to inlet port 20 and may terminate to volume reduction process at a preset pressure. In some embodiments where introduction of gas is driven by as constant driving pressure the volume reduction process may self-terminate at the point that the pressure drop across the filter reaches the driving pressure. When the suspension has been concentrated to the required degree valve 30 is opened and concentrate 56 may be unloaded along outlet fluidic pathway 28. Unloading of the concentrate may be by pressure from the second fluid the 37 via valve 38 or by a second filling of the first chamber with further suspension 50 via valve 34.

Suspension and the second fluid may be introduced into the first chamber using fluidic actuation means provided in the inlet fluidic pathways 35, 37, in sonic embodiments the fluidic actuation means comprise pump means such as pumps or syringes. In some such embodiment valves 34 and 38 are omitted. Concentrated suspension may be unloaded from the first chamber using fluidic actuation means provided in outlet fluidic pathway 29, for example by aspiration into a collection device such as a syringe. In embodiments in which the collection device is a syringe, the flow cut-if means is provided by the syringe plunger and valve 30 may be omitted, as shown in 1. FIG. 1h. In some embodiments the apparatus is adapted for manual operation, and the suspension and second fluid are introduced manually by means of syringes connected to fluidic pathways 35 and 37.

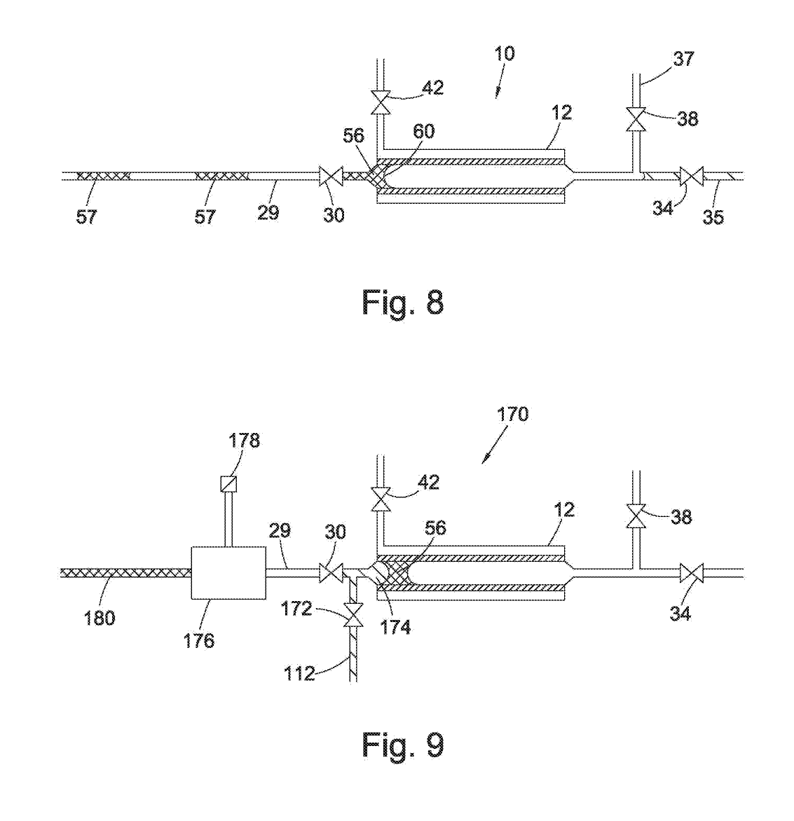

In this way the apparatus and method of the invention provides a single pass volume reduction process that may achieve a higher volume reduction ratio than prior art TFF processes, in a fluidic system comprising the fitter device having a lower ratio of total internal surface area to retentate chamber volume. In some embodiments the process may be a cyclical repetitive process controlled by operation of valves 30, 34 and 38 as described further with respect to FIGS. 1f, 1g and 8.

Referring to FIG. 1e a second embodiment of an apparatus of the invention comprises features as shown in FIGS. 1a-1d and further comprising a second inlet fluidic pathway 44 for the second fluid, connected to the outlet port 22, and controlled by a valve 46. In a method of use the second fluid is introduced into the first chamber via both port 20 and port 22, so providing a second meniscus 62, the suspension being concentrated between the two menisci.

According to the embodiment the immiscible fluid may be a gas. In some embodiments the immiscible fluid may be a liquid immiscible with the suspension, for example for an aqueous suspension, the immiscible fluid may be a non-aqueous liquid, and vice-versa. The gas may be air or another gas, chosen for example to maintain conditions within the suspension, such as an inert gas, an oxygen-free gas such as pure nitrogen, a gas containing oxygen or a gas containing carbon dioxide such as a CO₂/O₂/N₂ mixture to maintain the pH of a bicarbonate-buffered liquid, as for example in the case of a cell suspension. The gas may be humidified. The filter 14 is adapted such that in use when wetted with the first liquid of the suspension, the immiscible fluid does not pass through it. For example, for volume reduction of an aqueous suspension a hydrophilic fitter or a substantially hydrophobic fitter first wetted with aqueous liquid may he used, with the immiscible fluid being a gas, if the immiscible fluid is a non-aqueous liquid then a hydrophilic fitter is preferably used to avoid wetting of the filter with the non-aqueous liquid. The immiscible fluid will have a breakthrough or bubble pressure for passage through the filter into the second chamber. The apparatus and method are configured such that the maximum applied pressure across the filter is less than the breakthrough or bubble pressure. In the following description the immiscible fluid wilt be referred to as a gas, but it will be understood that similar principles will apply in the case of an immiscible liquid with modifications to the configuration of the apparatus as will be apparent to a skilled person.

In the description of the embodiments in FIGS. 1a to 1e and in ail embodiments herein, white the devices are depicted in the figures with the first chamber in substantially a horizontal orientation, no specific orientation of the device in use is implied. In some embodiments of methods of the invention the filter device(s) are oriented with the filter tilted away from horizontal, for example at an angle to horizontal, or with the fitter substantially vertical.

In some embodiments the filter is adapted to retain a first species while allowing a second to pass. It will be apparent that by selecting the type of filter used in the embodiments in FIGS. 1a to 1e, and in other embodiments herein, the apparatus can be made to retain and concentrate a first, larger species in the first chamber while a second, smaller species passes through the filter to the second chamber. For example, cells may be separated and concentrated while smaller particles such as lysed cell debris or viruses may pass through the filter.

Referring to FIG. 1f, an embodiment of an apparatus according to the invention comprises components as in the embodiment shown in FIGS. 1a to 1d and a control means 200 configured to control the valves 30, 34, 38 and optionally 42 by means of electrical control lines (dotted). The control means may also control fluidic actuation means not shown) configured to cause flow of suspension along inlet line 32 and gas along inlet line 36, and optionally along outlet line 28. The apparatus optionally comprises sensors adapted to read a condition in the apparatus, for example a pressure sensor 204 that reads the pressure in the first chamber 18, and a sensor 206 configured to sense the position of the meniscus 60, for example to sense when the meniscus has reached a chosen point within the first chamber. Such a sensor may operate for example on optical principles or ultrasonic principles, as in the manner of bubble detection sensors known in the art. The control means may read data from a data storage means 202 and may write data to it. Such data may compose instructions for operation of the apparatus and may comprise information including one or more of: sequence and timing of operation of the valves, for example how long each valve is open; readings from the sensors at which an operation is to be carried out for example the volume reduction process is to be stopped, information to control fluidic actuators connected to sources of suspension and gas to control flow along inlet lines 32, 36 and along outlet line 28. The data storage means 202 may comprise a remote data source and communication between the control means 200 and data storage means 202 may be via a data network. The data storage means may be physically associated with a vessel containing the suspension to be processed, and for example may comprise an electronic data storage chip, or a barcode, and the control means may be adapted to read such a data storage means. The control means may write data to the storage means derived from one or more sensors measuring conditions associated with fluids in the apparatus, such as for pressure, position of a meniscus, flow or temperature.

FIG. 1g shows an example of an operating sequence as may be carried out by the control means and steps of which may be stored in a data storage means as part of a control program for the control means. In an embodiment the control means may carry out the following operations in sequence:

Step 1: Optionally open valve 42 if it is previously closed.

Step 2: open valve 34: flow suspension into the first chamber. In this step a predetermined volume of suspension might be flowed into the first chamber so as substantially to fill the first chamber.

Step 3: close valve 30.

Step 4: close valve 34, open valve 38 and flow gas into the first chamber to effect volume reduction of the suspension.

Step 5: open valve 30 to unload the concentrated cell suspension under pressure from the gas supply.

Optionally, alternatively to step 5.

Step 5A: close valve 38 to close off the gas supply.

Step 6: open valve 30 and valve 34 to introduce a second volume of suspension into the first chamber. The second volume of suspension displaces the gas in the first chamber and forces the concentrated suspension through the outlet port 22 and into the outlet fluidic pathway 28.

In this way the apparatus may be used in a cyclical process in which a volume of suspension is introduced into the first chamber, concentrated, moved out from the first chamber and a second volume of suspension introduced into the first chamber. Such a cyclical process may be controlled by means of operation of the valves 30, 34 and 38 by the control means.

In an embodiment of an apparatus to carry out the process above the control means is configured to set the valves in the following three conditions;

a first condition in which the cut-off valve 30 is open, the suspension inlet valve 34 is open and the gas inlet valve 38 is closed;

a second condition in the cut-off valve 30 is closed, the suspension inlet valve 34 is closed and the gas inlet valve 38 is open, and

a third condition in which the out-off valve 30 is open, the suspension inlet valve 34 is closed and the gas inlet valve 38 is open.

In some embodiments the suspension inlet valve 34 is a check valve configured to allow flow into the inlet fluidic pathway 26 when there is a forward pressure drop across the valve, i.e. the pressure in suspension supply pathway 35 is greater than the pressure in net fluidic pathway 26. A method for cyclical volume reduction may then be implemented by means of control over valves 30 and 38. Gas pressure Pg in gas supply pathway 37 is chosen to be greater than suspension supply pressure Ps in suspension supply pathway 35, and the pressure Po in the concentrate output fluidic pathway 29 is less than Ps and Pg and may be substantially atmospheric pressure, in one embodiment steps in the method are

Step 1: open valve 30 and close valve 38. The first chamber is now at Po, check valve 34 opens and the first chamber fills with suspension.

Step 2: close valve 30 and open valve 38. The first chamber is now pressurised to Pg, check valve 34 closes and gas is introduced into the first chamber, forcing liquid from the suspension through the filter and concentrating the suspension.

Alter a chosen time for gas introduction, or a chosen volume of gas has been introduced, or under sensor control as described above, repeat step 1. As check valve 34 opens and the first chamber fills again with suspension, concentrated suspension is expelled from the first chamber through valve 30 into output fluidic, pathway 29.

In this way in some embodiments step 1 and step 2 form a repeating cyclical process in which aliquots of the suspension are introduced into the first chamber, concentrated and then flowed out along the output fluidic pathway,

In some embodiments of the method, a fluidic actuation means provided in outlet fluidic pathway 29 is actuated to unload the concentrated suspension.

An embodiment of an apparatus configured to carry out the process comprises an apparatus as described with respect to FIGS. 1a-1d and a control means configured to control valves 30 and 38 such that when valve 30 is open, valve 38 is closed and vice versa. Flow actuation of the suspension and the gas may be for example by pressurisation of reservoirs of suspension and gas, such as by pressurising as gas headspace above the suspension.

Accordingly, in an embodiment of an apparatus comprising a check valve 34 the control means is configured to set valves 30 and 38 in the following conditions:

a first condition in which the cut-off valve 30 is open and the gas inlet valve 38 is closed (the suspension inlet check valve 34 is open in this condition);

a second condition in which the cut-off valve 30 is closed and the gas inlet valve 38 is open (the suspension inlet check valve 34 is closed in this condition).

In some embodiments the control means is configured to set valves 30 and 38 in a further condition in which the cut-off valve 30 is closed and the gas inlet valve 38 is also closed (the suspension inlet check valve 34 is closed in this condition).

The filter devices in the embodiments of FIGS. 1a to 1f may comprise a tubular vessel 11 formed for example within a solid body and having an interior wall 13, the vessel being divided by filter 14 into a first chamber 18 and a second chamber 16. The vessel may be formed as defined previously, according to the embodiment. The vessel may have a shape, for example having one of a rectilinear, triangular, oval, circular, semi-oval or semicircular cross-sectional shape perpendicular to a long axis of the vessel and one of a rectilinear, triangular or oval cross-sectional shape parallel to the long axis of the vessel.

The vessel 11 may taper towards the inlet port having a tapering profile 21 at the inlet end and 23 at the outlet end, or both, such that the first chamber tapers towards one or both ports. According to the embodiment, such a taper may take various forms as defined previously. In some embodiments the vessel has substantially a constant cross-sectional area, and in some embodiments the first chamber is substantially the same cross-sectional area as the inlet or the outlet ports.

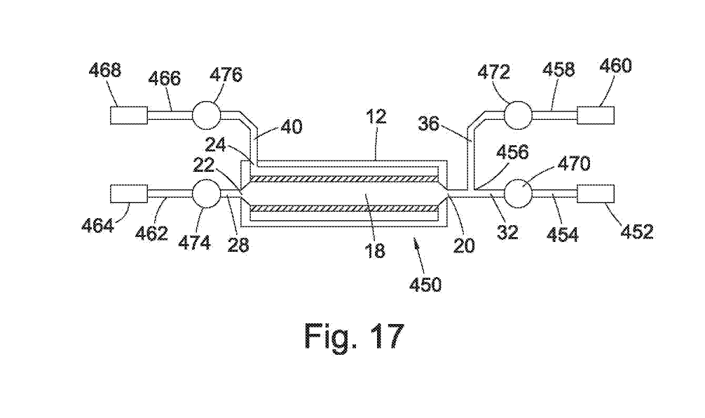

The filter 14 may be a substantially cylindrical filter or may be formed from two or more planar filters stacked to form one or more first chambers 18, separated by the filters from one or more second chambers 18 as shown in FIGS. 17 and 18. In some embodiments the first chamber may comprise a single planar filter and an opposing impermeable wail.

Referring to FIG. 1h, an embodiment 45 comprises features as shown in FIGS. 1a to 1d and further comprising a collection device in the form of a syringe 120 detachably connected to the outlet port 22 of the device 12 by means of a detachable connection 121. The detachable connection may be a fluidic connection as known in the art such as a Luer connection. In this way a suspension may be concentrated in the first chamber 16 in the manner shown in FIGS. 1a to 1d and then unloaded by aspiration into the syringe 120. The flow cut-oft means in this embodiment comprises the syringe itself, the plunger being optionally held in place against movement while the suspension and second fluid are introduced into the first chamber, for example by a syringe actuator engaged with the plunger. In an alternative embodiment the syringe 120 is omitted and the outlet fluidic pathway 28 comprises a septum. Unloading of concentrate suspension may then be done by means of a syringe and needle to pierce the septum. Such embodiments are adapted for example for processing a cell suspension such as a cell therapy suspension, the concentrated suspension being provided within the syringe ready for administration to a patient The apparatus may comprise means to introduce a wash liquid into the first chamber in the manner of other embodiments herein.

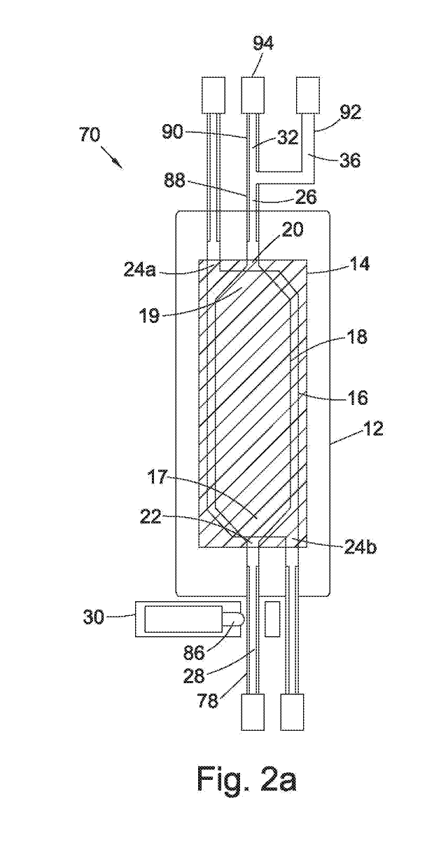

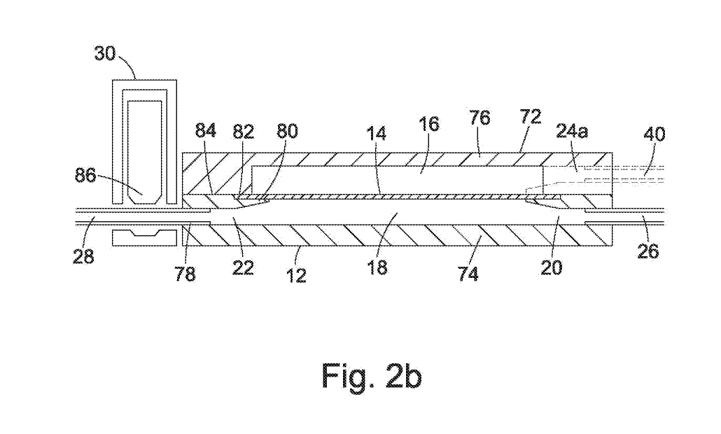

Referring to FIGS. 2a and 2b, a further embodiment 70 of an apparatus according to the invention comprises a filter device 12 and a cut-off valve 30 as described before. The filter device comprises a housing 72 comprising a lower housing component 74 and an upper component 76, the two being adapted to retain and to seal to the filter 14 disposed between them. The first chamber 18 has a substantially rectilinear shape having a length, a width less than the length, and a depth less than the width, a long and a short axis, the inlet port 20 and outlet port 22 being disposed distally in the direction of the long axis. Conveniently the first chamber tapers towards the net and outlet ports across its width as shown in FIG. 2a and also optionally across its depth as shown in FIG. 2b, so having an outlet taper region 17 and an inlet taper region 19. In this way the first chamber provides a useful area of filter 14 while providing a flow pattern at the inlet and outlet that reduces dead space and aids filling and draining of the first chamber, in this embodiment the filter covers the taper regions 17, 19. In some embodiments a region of the first chamber adjacent to the outlet port 22 does not have fluid communication through the filter, for example the filter stopping short of the outlet port 22, or the second chamber stopping short of the position of the port, thereby forming a dead stop region of the first chamber adjacent to the outlet port in which suspension is not longer concentrated. This allows the apparatus in some embodiments to perform a self stopping volume reduction process as will be described in detail with respect to later embodiments.

The second chamber 6 is provided with a first outlet port 24a and an optional second port 24b at opposing ends of the second chamber, the optional second port to assist in priming the second chamber. The shape of the second chamber is shown as being substantially rectilinear and overlapping the first chamber 18 so as to avoid dead spaces around the edge of the first chamber no fluidic connection through the filter to the second chamber.

Sealing means are provided at the seal area 80 between the filter and the lower component and seal area 82 between the filter and the upper component. Such sealing means may comprise an elastomeric seal such as an O-ring, or in some embodiments the fitter may be bonded to one of the upper and tower housing components, for example by ultrasonic bonding, heat sealing, or adhesive bonding, depending on the filter material as known in the art. Housing components 74 and 76 may be bonded together in their contact area 84, or may be held together for example by bolts or staking. The device may be configured to be capable disassembly, so that the fitter may be removed or replaced. The filter may be provided having a filter material seated into a surrounding frame, the frame and device components being adapted to form a seal between them when the device is assembled. Fluidic connections may be merle to the device by tubing 78 defining for example outlet fluidic pathway 28, and may in alternative embodiments be formed at feast in part by channels within an extension of the housing 72.

In the embodiment 70 at least the outlet fluidic pathway 28 defined at east partially in a collapsible elastomeric tubing and the cut-off valve 30 is a pinch valve, having a pinch valve actuator 86 adapted to close the outlet tubing. In FIG. 2b valve 30 is shown perpendicular to the minor axis of the first chamber 18 and in FIG. 2a it is shown as parallel to it; these represent alternative arrangements of the out-off valve that may be provided in different embodiments but the function is the same. Valve 30 is shown separate from the housing 72 for clarity but in some embodiments valve 30 is mounted on or forms part of the housing. In some embodiments valve 30 is a manifolded cut-off valve mounted on one of the lower or the upper housing components, the outlet fluidic pathway 28 comprising a fluidic channel formed within or defined by one or both components and connected to the outlet port 22 through the valve.

Fluidic connection to the apparatus may be made by means of flexible tubing 78, 88. The inlet fluidic connection 88 may branch into a suspension inlet 90 and a second fluid inlet 92 as shown. In some embodiments the first chamber comprises two inlet ports having a separate fluidic connection to eat. Interface to further fluidic components is optionally made using fluidic connector means, for example Luer connectors. In some embodiments further fluidic components may be integrated into the housing 72, for example reservoirs and fluidic actuation means.

The embodiment 70 may comprise a housing 72 the form of a rigid body component comprising upper and lower components 74, 76, and may form part of an apparatus comprising further fluidic pathways and component formed on or within the body component. For example the apparatus of FIGS. 1a to 1f, 2a, and 2b may form a sub-unit of a larger apparatus such as a processor as described herein, may be detachably mounted on the processor, the processor comprising a plurality of apparatus according to the invention, optionally within a common housing. In some embodiments the housing 72 comprises one or mare flexible of semi-flexible components and may be formed in the manner of a beg, the upper and lower components both being formed from flexible polymer material, the filter 14 being formed from a flexible membrane, and being sandwiched between the upper and lower housing components, the components being sealed together by known sealing means such as heat sealing. The inlet and outlet ports may be formed by the openings to tubes inserted between and seated to the upper and lower components.

In some embodiments the apparatus composes a device 12 having tubular connectors a processor onto which the device mounts, the processor comprising the pinch valve actuator 86.

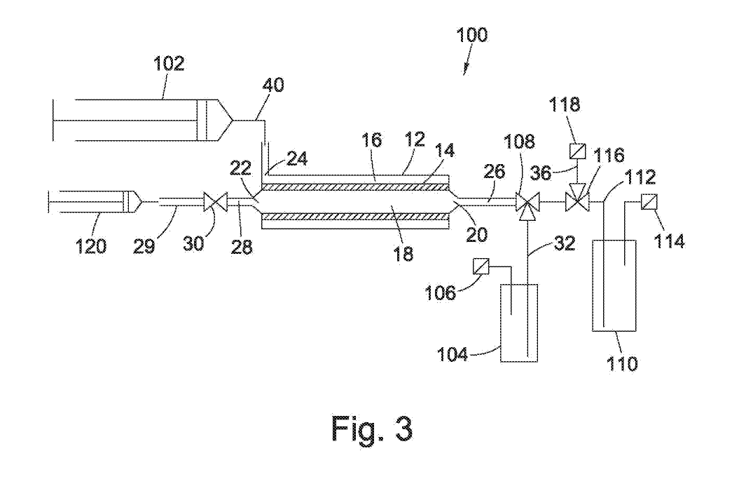

Referring to FIG. 3 an embodiment 100 of an apparatus according to the invention comprises a filter device 12 having common features as described for the embodiment in FIG. 1a to 1d with Common numerals. In this embodiment the second immiscible fluid is gas such as air and the source is an atmosphere in gaseous communication with the gas inlet fluidic pathway 36 through a filter means 118 and valve means 118, here shown as a 3-way valve but which in some embodiments may comprise two 2-way valves. In this embodiment the filtration process is driven by a pressure at the filtrate port 24 that is less than atmospheric pressure, generated by syringe 102, which also serves as a filtrate container. Alternatively a filtrate container may be provided connected to filtrate port 24, a negative pressure in the filtrate container being created by a pump or syringe means; or a pump means may be connected to filtrate port 24 and the pump outlet be connected to a filtrate container. The apparatus further comprises a source of a third liquid such as a wash liquid miscible with the first liquid in the form of a reservoir 110, in fluid communication with the inlet port 20 via third liquid inlet fluidic pathway 112 and valve means 116. The filter device is in fluid communication with a collection device 120 via outlet port 22 and cut-off valve 30.

In some embodiments cut-off valve 30 is a check valve adapted to allow flow in the direction from port 22 outwards along fluidic pathway 28, but not in the reverse direction.

A system comprising an apparatus according to the invention comprises a source 104 of liquid containing a species in suspension in the form of a reservoir 104, in fluid communication with the inlet port 20 of the fitter device. Reservoir 104 is connected to the suspension inlet fluidic pathway 32, and to the filter devise inlet fluidic, pathway 26 via valve means 108, again shown as a 3-way valve but which may compose two 2-way valves.

A method for filtration of a first liquid containing a species in suspension using the apparatus 100 and a system composing the apparatus comprises the following steps:

(i) causing a first liquid containing the species to flow into the first chamber by means of a pressure below atmospheric pressure at the filtrate port 24, created by syringe 102, from the reservoir 104 via valve means 108.

(ii) introducing a gas into the first chamber via port 20 white the fluidic pathway from the outlet port 22 is closed, while liquid flows through the filter to the second chamber, to form a concentrated suspension containing the species in the first chamber,

and subsequently

(iii) flowing the concentrated liquid containing the species in suspension formed in step (ii) out from the first chamber to the collection device 120.

In this embodiment concentrated suspension is flowed out from the first chamber by aspiration by the syringe 120. Pressure in the first chamber may be equalised during unloading by air entering inlet per 20 from breather 118.

In some embodiments the method further comprises the step of controlling the cut-off valve 30 to open it before step (iii) above.

In embodiments in which the cut-off valve 30 is a check valve, during steps (i) and (ii) the pressure in the first chamber 18 is maintained less than the pressure in fluidic outlet pathway 29 to the collection device, so check valve 30 is closed. During step (iii) the pressure in fluidic outlet pathway 29 is lower than that in the first chamber, so check valve 30 opens, allowing concentrated suspension to flow out along outlet fluidic pathway 28 and through the valve.

In some embodiments the method composes the further step of causing a third liquid to flew into the first chamber from reservoir 110. In this way a wash liquid may be flowed through the suspension to wash an undesired component out of the suspension into the second chamber and thence to syringe 102.

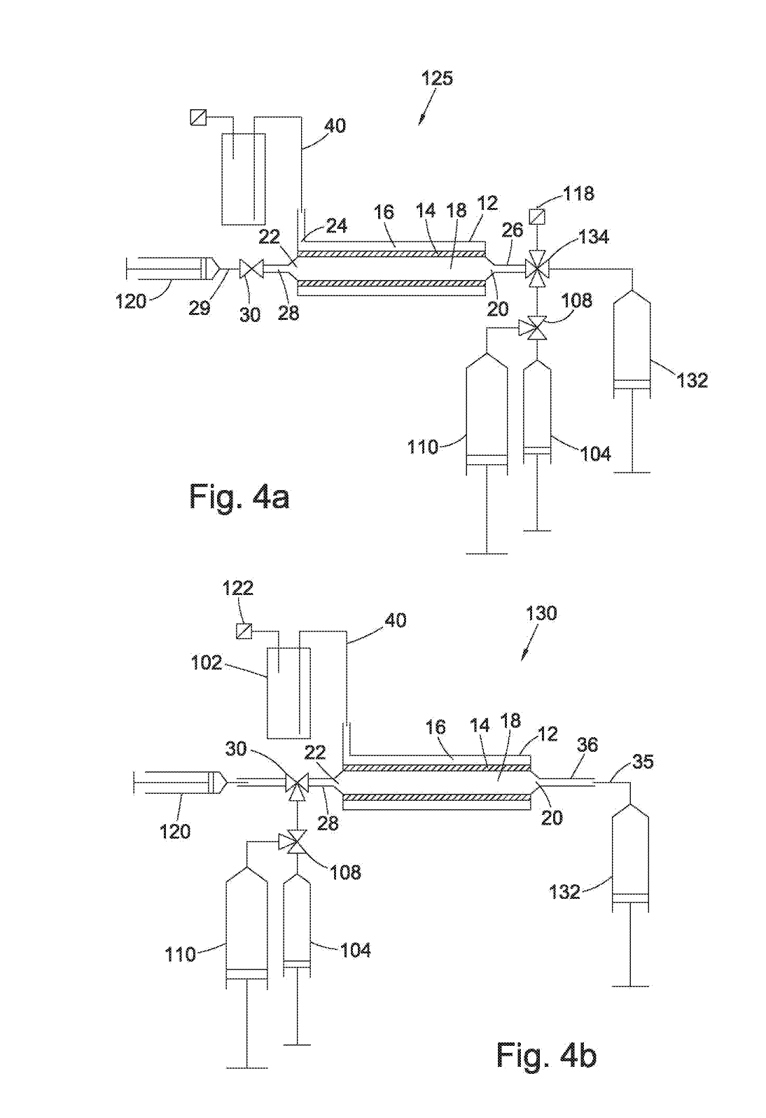

Referring to FIG. 4a, a further embodiment 125 of an apparatus according to the invention composes a fitter device 12 as before and a out-off valve 30, the apparatus further composing a source of gas in the form of a reservoir 132, here shown as a syringe, connected to port 20 of the device via valve 134. Sources of suspension 104 and third liquid 110 such as a wash liquid are connected to port 20 via valve 108 and valve 134. In this embodiment, suspension and the third liquid are introduced into the first chamber through port 20 by means of positive pressure from syringes 104 or 110. Filtrate outlet pathway 40 may be dosed by a valve (not shown) in some embodiments. In some embodiments slugs of suspension and third liquid may be introduced alternately during the filling process by means of valve 108. Optionally the suspension and the third liquid may be mixed by a to-and-fro movement of the slugs through the first chamber by means of syringe 110. The suspension in the first chamber is then concentrated by flowing gas from syringe 132 into the first chamber. Concentrated suspension may then be flowed out a the first chamber via valve 30 to the collection device 120, again shown in the form of a syringe. Optionally valve 134 is a 4-way valve having a fluidic pathway to a breather 118 as shown in FIG. 4a. Pressure equalisation in the first chamber during unloading may be achieved by opening valve 134 to breather 118. Alternatively valve 134 may be a 3-way valve, breather 118 may be omitted and pressure equalisation may be either through the filter or by means of gas supplied from gas source 132. Repeated cycles of introduction of third liquid from reservoir 110, mixing and volume reduction may be carried out in order to give effective washing of the suspension.

It will be apparent that in sonic embodiments in which the collection device 120 comprises a flow cut off means that prevents flow along outlet fluidic, pathway 28, 29, such as a syringe with the plunger held in place by an actuator mechanism or other means, valve 30 may be omitted, as shown in FIG. 1b. The flow but-off means in the outlet fluidic pathway is then provided as part of the collection device.

Referring to FIG. 4b, a further embodiment 130 of an apparatus according to the invention comprises a filter device 12 as before and a cut-off valve 30 here in the form of a 3-way valve having a position in which the fluidic pathway 28 from the port 22 of the filter device is closed, the apparatus further comprising a source of gas in the form of a reservoir 132, here shown as a syringe, connected to port 20 of the device. Sources of suspension 104 and third liquid 110 are connected to port 22 via 3-way valves 108 and 30. In this embodiment, suspension and the third liquid are introduced into the first chamber through port 22, and optionally may be mixed by flowing the slugs within the first chamber by means of syringes 132, 110.

It will be apparent from the foregoing discussion that the apparatus of the invention in some embodiments benefits from a close connection of the cut-off valve to the test point of contact of the fitter with the fluidic pathway through the first chamber. Suspension that occupies the volume between the filter and the cut-off valve is not in contact with the filter and so is likely not to undergo volume reduction by introduction of immiscible fluid. Efficiency of volume reduction is therefore increased if that volume is reduced. Therefore in some embodiments the apparatus comprises a close connection as defined herein between the port 22 and the cut-off valve 30. Referring to the outlet dead volume as the volume in the fluidic pathway between the pod 22 and the valve 30, a close connection between the port and valve means that the outlet dead volume is less than the volume of the first chamber, in some embodiments between 0.1 and 1.0 times the volume of the first chamber, in some embodiments between 0.02 and 0.2 times the volume of the first chamber and in some embodiments between 0.005 and 0.05 times the volume of the first chamber. In some embodiments the filter may only be provided in a portion of the wall of the first chamber. Therefore in some embodiments, referring to the active volume of the first chamber as the volume within which the filter surface intersects a line perpendicular to the centroid of the fluid flow pathway through the first chamber, the outlet dead volume is less than the active volume, in some embodiments between 0.1 and 1.0 times the active volume, in sonic embodiments between 0.02 and 0.2 times the active volume and in some embodiments between 0.005 and 0.05 times the active volume.

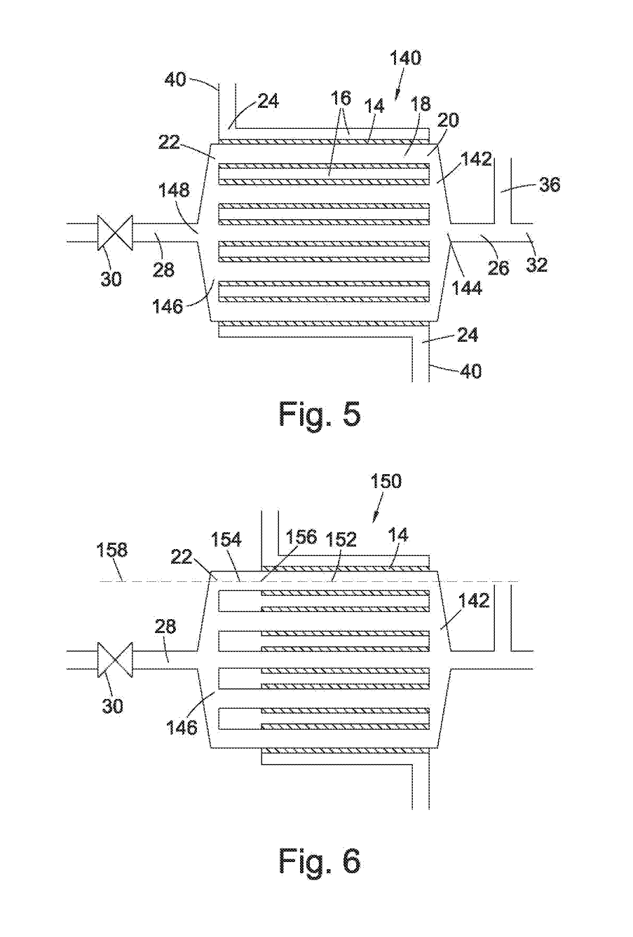

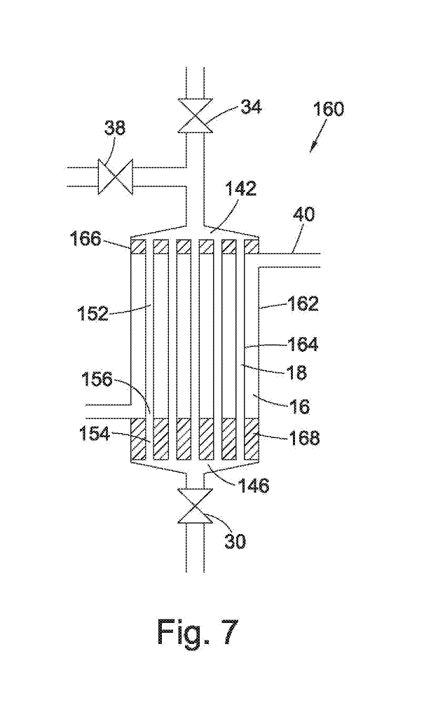

Referring to FIG. 5, an embodiment 140 of a filter device adapted for use as part of the apparatus is shown diagrammatically, having a plurality of first chambers 18 in fluid communication with an inlet fluidic pathway 28 via, an inlet manifold 142 and with an outlet fluidic pathway 28 via an cutlet manifold 146. Each first chamber is in fluid communication with a second chamber 16, which may be a common second chamber in fluidic communication with all of the first chambers. The second chamber may have one or more outlet ports 24 leading to one or more outlet fluidic pathways 40. In this way suspension and then gas may be introduced to the device 140 via a manifold inlet port 144 and distributed to the plurality of first chambers 18. The volume reduction and a wash process using a third liquid may be carried out in the first chambers as described previously, with control of flow out from the device along outlet fluidic pathway 28 by means of cut-off valve 30. In some embodiments filters 14 comprise tubular fitter membranes sealed into a housing as known in the art, for example in the form of a hollow fibre fitter cartridge as described with reference to FIG. 7. In some embodiments the filters 14 comprise a stack of substantially planar filter membranes spaced apart, in which case the diagram in FIG. 5 may show a vertical cross section through a stack of substantially horizontal filter membranes or a horizontal cross section through an array of substantially vertical filter membranes.

Referring to FIG. 6, an embodiment 150 of a filter device is similar to that of FIG. 5 but in which the filters 14 extend only partially along the length of the first chambers 18, the first chamber comprising an active volume 152 as defined herein, shown hatched in FIG. 6, and a concentration volume 154, separated by the position 156 defined by the point at which the filter ceases to intersect a line perpendicular to the centroid of the fluidic pathway through the first chamber indicated by the axis line 158 in FIG. 6. The volume in the outlet manifold 146 and in the outlet fluidic pathway 28 before the valve 30 forms the outlet dead volume, as described above.

In use, during the volume reduction process, the meniscus will advance in each of the first chambers 18 independently, and may advance at different rates in each first chamber. This may happen if the first chambers are small and there is significant liquid flow resistance within them; as gas enters the first chambers, resistance to liquid flow from the remaining shorter liquid column will be reduced, leading in principle to a situation where one or more first chambers 18 may empty before the others. When the meniscus reaches position 156 no further liquid can exit via the fitter 14, and so in the embodiment in FIG. 6 the concentration volume acts in use to form a residual volume in each of the first chambers within which the suspension will not be concentrated, so acting as to control the minimum volume of the concentrated suspension in each of the first chambers when the volume reduction process is terminated, in this way the risk is reduced that a gas introduced into the first chambers 18 in parallel during volume reduction will empty one of the first chambers completely, so allowing gas to enter the outlet manifold 146, where it may cause a gas-lock and prevent correct functioning of the device, in particular hindering unloading of concentrated suspension.

Referring to FIG. 7, an embodiment 150 comprises a filter device in the form of a hollow fibre filter cartridge 162 comprising a plurality of hollow fibres 164 in a housing 166 and sealed in a seal layer 158, defining a plurality of first chambers 18 within the lumens of the fibres and a common second chamber 16 surrounding them. Such an embodiment may be used as described with reference to FIGS. 5 and 6, and the cartridge may be adapted in some embodiments to provide a concentration volume 154 within the fibres in the region in which they are sealed into the housing.

Referring to FIGS. 6 and 8, repeated cycle volume reduction and washing of a suspension is further illustrated. In FIG. 8 an embodiment 10 comprises components as in FIGS. 1a to 1d. Repeated cycles of introduction of suspension and then gas into the first chamber results in slugs 57 of concentrated suspension being formed and then flowed out from the first chamber via cut-off valve 30, with slugs of the gas introduced in the volume reduction steps separating them. As shown in FIG. 9, an embodiment 170 comprising a debubbler 176, for example with a gas vent through breather 178, in the fluidic output pathway 29 removes the gas to provide a substantially continuous concentrate phase 180 that may be flowed along outlet pathway 29 or collected within the debubbler, in which case the latter might comprise simply a vented container. The embodiment 170 is further configured to add a third liquid to the concentrate, for example to wash out an undesired component from the suspension or to add a desired component. A source of wash liquid is connected to the wash liquid inlet pathway 112 and thence to port 22 of the filter device via valve 172. Wash liquid is conveniently supplied to the outlet end of the first chamber as this is where the concentrate will reside following volume reduction. Following a first volume reduction step, wash liquid 174 may be flowed into the first chamber and optionally mixed with the adjacent concentrate slug 56 by a process of moving the two liquids to and fro within the first chamber and/or connected fluidic pathways. The volume reduction step may then be carried out again. This process may be done in sequence to wash the suspension. The degree of volume reduction in each step may be chosen to allow efficient mixing. In a final volume reduction step the degree of volume reduction may be chosen to be greater to provide a concentrated output.

Such an embodiment might be used for example in processing of a cell suspension, for example to wash out a cryoprotectant such as DMSO and to introduce an excipient, for example PBS or saline, for administration to a patient.

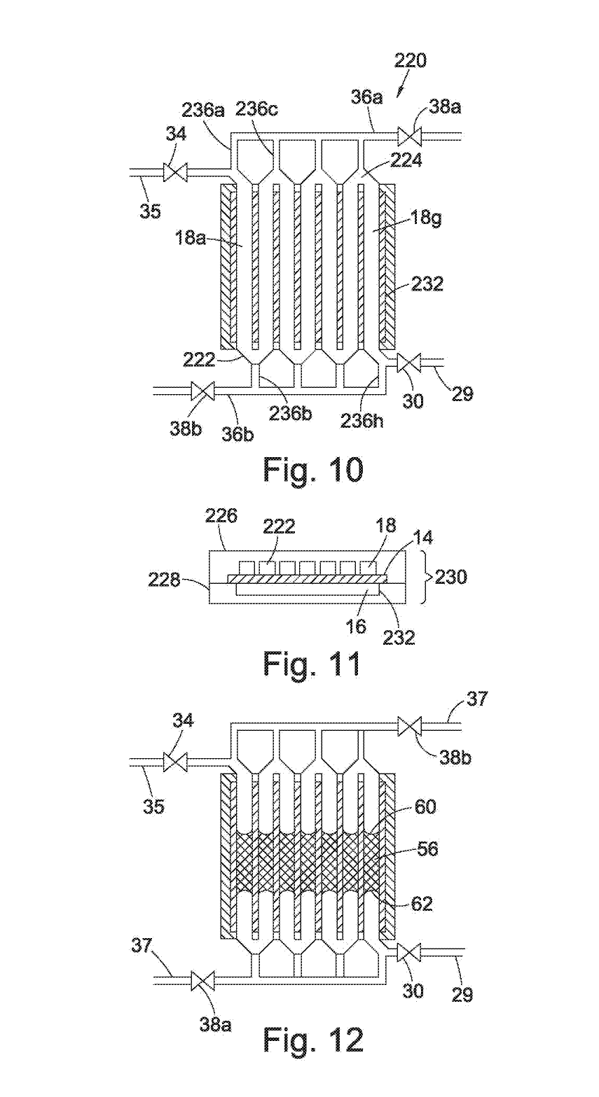

Referring to FIGS. 10 to 12, a further embodiment of an apparatus according to the invention comprises a filter device 220 comprising an elongated channel 222 defining together with a planar filter 14 a first chamber 18 comprising a plurality of first chamber portions 18a to 18g in series along the channel, and a second chamber 18 defined by a recessed region 232 together with the filter 14. The channel 222 is conveniently in a serpentine form such that several areas of the channel lie adjacent one to another over the filter, so defining a series of first chamber portions 18a to 18g joined by curved linker regions 224. This is an efficient means of providing an elongated filter area over a channel while using a tow aspect ratio and easy to handle filter component. In this embodiment the device 220 is configured to provide gas inlet positions at a number of places along the length of the first chamber, namely at the linker regions 224 between the first chamber portions. Gas inlet fluidic pathways 36a and 36b are shown as being controlled by air inlet valves 38a and 38b though it is understood that the gas inlet pathways may be common and may be controlled by a single valve. Gas inlet sub-pathways 236a to 236h provide a gas inlet at each end of each first chamber portion.

The device 220 may be formed for example in a housing 230 comprising an upper portion 226 comprising the channel 222 and lower portion 228 comprising the recess 224 forming the second chamber, the two portions being adapted to seal a planar filter element 14 such as a filter membrane between them. The housing portions may be formed by moulding, embossing or machining in polymer and joined by for example welding, ultrasonic welding, heat seating, solvent welding, adhesive bonding, snap fit joining, staking or bolting together as known in the art.

In use, as shown in FIG. 12, the first chamber may be filled with suspension, then gas may be introduced into the first chamber through gas inlet sub-pathways 236a to 236h with valves 30 and 34 closed, so creating a first and a second meniscus 60, 62 in each first chamber portion, so effecting volume reduction in each first chamber portion. The concentrate may then be flowed out via valve 30 into outlet fluidic pathway 29 as a series of slugs of concentrated suspension.

In some embodiments the invention is directed to devices, apparatus and methods for processing a suspension by adding a component to the suspension. Such embodiments are particularly adapted for adding a component such as a cryoprotectant to a cell suspension. Such cryoprotectants are often needed in high concentration, for example in the range over approximately 5% and in some cases over approximately 10% of the suspension by volume. Ctyoprotectants may contain compounds such as dimetnyt sulphoxide ipMS0) that are toxin to cells which are metabolically active and so need to be added carefully and at low temperatures such as in the range 2 to 8 degrees C. Addition of cryoprotectant, in particular containing DMSO, also often results in an exothermic reaction that means that cryoprotectant needs to be added slowly and uniformly, for example with good mixing and the mixture needs to be continuously cooled, for example on ice, in present manual practice a volume reduction step is usually carried out, for example by centrifugation, followed by dropwise addition of the cryoprotectant with mixing and cooling, resulting in a laborious and time consuming process. The volume reduction devices and apparatus of the invention are well adapted to volume reduction of a cell suspension and may be extended with features adapted to provide effective and labour-saving addition of cryoprotectant to the resulting concentrate. A source of cryoprotectant may be connected to the outlet fluidic pathway 29 downstream of the cut-oft valve 30 in the embodiments described herein, and cryoprotectant and the concentrate may be flowed together into, arid may he mixed within, the outlet fluidic pathway or a mixing chamber provided therein. A mixing chamber may be for example a chamber of a larger cross sectional dimension than the fluidic pathway 29, so encouraging the concentrate and the cryoprotectant to mix A mixing chamber may comprise features on its interior surface such as grooves to promote mixing, as known in the art.

Embodiments as shown diagrammatically herein may comprise a tubular first chamber 18 defined by tubular fitter, such as a rigid cylindrical fitter element, a tubular filter membrane such as a cylindrical membrane, or may be defined by one or more planar filters together with one or more impermeable structural members.

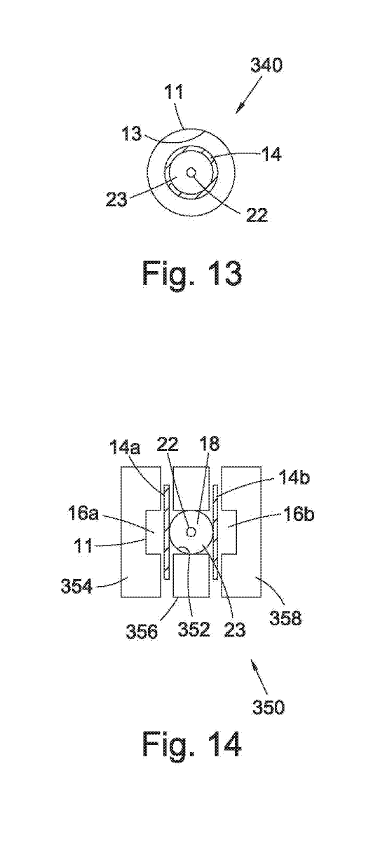

Referring to FIGS. 1a to and 13, an embodiment 340 of a filter device according to the invention shown diagrammatically in axial cross-section in figure to is shown in radial cross-section in FIG. 13, and comprises a tubular for example cylindrical vessel 11 having an inner well 13 and a tubular, substantially cylindrical fitter 14. The first chamber is defined within the filter and the second chamber is defined external to the filter. The vessel comprises a wall 23 tapering towards the outlet port 22. It will be understood that a filter device may comprise a plurality of tubular filters in a common vessel 11, providing a plurality of tubular first chambers within them.

Referring to FIGS. 1a and 14, an embodiment 350 comprises a vessel comprising first 14a and second 14b planar filters, divided into a first chamber 18 and second chamber portions 16a and 16b in fluid communication with the first chamber via filters 14a and 14b respectively. The second chamber portions 16a, 16b are connected to each other either within the device or by a fluidic pathway provided as part of an external fluidic system. In an exemplary construction the filters 14a, 14b are mounted within body components 354, 358, 358 as shown in FIG. 14, the vessel 11 and the first chamber and second chamber portions being defined by recesses and apertures formed within the body components, the body components being assembled together for example by bonding or fixings as described previously. The first chamber tapers towards the outlet port 22 and may comprise a frustoconical portion having a tapering surface 23 with a circular inner profile 352. It will be understood that a filter device may compose a plurality of stacked planar filters separated by spacers to define a series of first chambers 18 separated by second chambers 16, which in some embodiments are portions of a common second chamber space. In this way the device may have a configuration and function as described with reference to FIGS. 5 and 6.

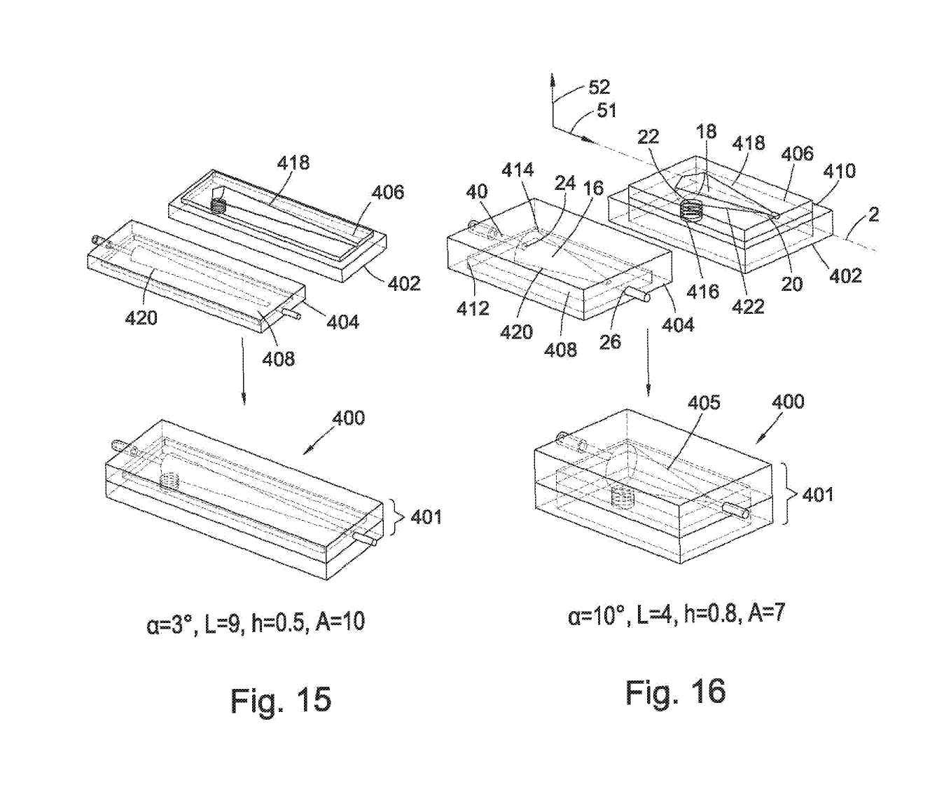

Referring to FIGS. 15 and 16, filter devices usable in apparatus according to the invention are shown, the two devices each having features described for the other embodiments but being of different geometry. In each case the filter device 400 comprises a body component 401 comprising a lower body part 402 and an upper body part 404 adapted to fit together so as to define a vessel 405 composing the first chamber 18 and the second chamber 18, that are separated by a filler membrane 14 (not shown) when the body parts are assembled. The body parts are adapted to be bolted together via holes (not shown). The lower body part has an upper surface 406 here provided on an upstand 410, adapted to fit together with a lower surface 406 on the upper body part provided in a recess 412. The first chamber 18 is formed as a recess in the upper surface 406, having a perimeter 418 in the surface, and the second chamber 16 is formed as a recess in the lower surface 408, having as perimeter 420 in the surface. The body pads are adapted to retain a fitter membrane (not shown) between them and each to form a liquid tight seal to the filter membrane outside the perimeters 418, 420 when the body components are held together.

The first and second chambers are each in the farm of a longer cone and a shorter cone joined at their bases, split across the diameter of the cones. The filter area between the chambers is substantially a kite shape as shown by the perimeter 418. The inlet port 20 and outlet port 22 are provided as openings to the wall of the first chamber 18, and the first chamber tapers towards both the inlet port 20 and the outlet port 22. The lower wall 422 of the first chamber is at an angle to the plane of the fitter, here referred to in the figure captions as alpha. The outlet port 22 opens to the will of the first chamber at the join of the two cones, that is at the lowest point of the first chamber. The outlet port is provided as part of a threaded recess 416 adapted to receive a mate thread Luer connector via which a collection device such as a syringe may be connected, optionally via a out-off valve. Fluidic pathways 26 to port 20 and 46 from port 24 are formed as channels within the body parts.

In this way the embodiment 400 has the following characteristics and functions:

The first chamber tapers towards the inlet port 20—this facilitates the meniscus entering from port 20 to expand gradually during the volume reduction process having a profile governed by the contact angle on the filter and on the lower wall 142.

The first chamber tapers towards the outlet port 22—this facilitates the concentrated suspension being collected in the tapering region close to the outlet port, so allowing efficient flow of the concentrate out through the outlet port.

The device is adapted to permit a flow cut-off means such as a cut-off valve, or a collection device such as a syringe, to be connected to the outlet port with low outlet dead volume. In some embodiments the connection may he a close connection as defined herein.

The geometry and dimensions of the example filter devices are shown in the captions to FIGS. 15 and 16. Alpha (degrees) is the angle of the longer cone, which is also the angle of slope of the lower wail 422 to the plane of the filter, i.e. also the angle of the lower wall 422 to horizontal if the device is oriented with the filter horizontal. L (cm) is the total length of the two cones and hence of the long axis of the filter, h (cm) is the height of the first chamber perpendicular from the plane of the surface 406 to the circumference of the base of the cones, which is this example lies within port 22, and A (cm2) is the total area of the filter membrane between the two chambers defined by perimeters 418 and 420. The devices shown in FIGS. 15 and 16 both have a volume in the first chamber below the plane of surface 408 of 1.5 ml. As is shown, for a smaller angle of slope alpha the device is longer and shallower to achieve the same first chamber volume.

Referring to FIG. 17, in a further embodiment the invention provides an apparatus comprising a fluidic unit 450 and a processor, together configured to carry out a process within the fluidic unit, the fluidic unit comprising a filter device as described herein and being adapted to interfit with the processor, the processor composing one or more actuators configured to control fluid flow within the fluidic, unit and a control means configured to control the actuators,

The fluidic unit 450 comprises:

a filter device 12 as described herein,

an inlet 452 adapted to receive a suspension,

a port 20 opening to a first chamber 18 of the filter device,

a fluidic pathway 32 connecting the inlet to the port,

a fluidic pathway 32, 36 configured to introduce a second fluid to the first chamber, and

an outlet fluidic pathway 28 leading from a port 22 opening to the first chamber,

wherein the fluidic pathways are provided within flexible tubing bonded to the filter device to form a tubing set comprising the filter device.

The tubing set composes:

a tubing portion 454 connecting inlet 354 to port 20 providing fluidic pathway 32;

a tubing portion 458 connecting second fluid inlet connection 460 and port 20, providing fluidic pathway 36;

a tubing portion 462 connecting pod 22 to outlet connection 464 and providing fluidic pathway 28;

a tubing portion 466 connecting filtrate port 24 to filtrate connection 468 and providing fluidic pathway 40.

The tubing portions, junctions and other components such as the connectors may be joined by means known in the art, for example tube welding or using connection means such as Luer connectors.

The fluidic unit 450 is adapted to be mounted on a processor such that tubing portions 454, 458, 462 and 466 interfit with valve actuators 470, 472, 474, and 476 respectively, provided on the processor, to form a pinch valve in each of the said tubing portions. In this way the fluidic unit 450 and the processor together form a cutoff valve au in the outlet fluidic pathway 28 from port 22, comprising tubing portion 462 and actuator 474, and also valve 34 in suspension inlet pathway 32, valve 38 in second fluid inlet pathway 36 and valve 42 in filtrate outlet fluidic pathway 40 in the manner shown in figure is by means of the corresponding tubing portions combined with pinch valve actuators.

It will be understood that in some embodiments the fluidic unit 450 may comprise further components as shown for other embodiments herein, such as a collection device connected to fluidic pathway 28, a waste reservoir connected to fluidic, pathway 40, a source of a second fluid connected to second fluid inlet pathway 36, and a source of a wash liquid may be connected to for example inlet 452 or to a further fluidic pathway as shown in other embodiments. The inlet 452 may comprise an aseptic connection as known n the art, for example a septum. In this way the fluidic unit 450 may form a closed system for processing a suspension in aseptic conditions, movement of fluids within the fluidic unit 450 being actuated and controlled by the processor.

According to the embodiment the filter device forming part of the fluidic unit 450 may take one of the forms described herein. For example in one embodiment the device is a hollow fibre fitter cartridge. According to the embodiment the fluidic unit may comprise a fluidic system or arouse as described for the embodiments herein without being limited to the arrangement shown in FIG. 17.

The invention will now be further described by way of reference to the following Example which is present for the purposes of illustration only and is not to be construed as being a limitation on the invention. A method as described herein for embodiments 100 and 125 was used with a filter device 400 as shown in FIG. 16 forming part of an apparatus 125 as shown in FIG. 4a. The concentrate was recovered from the first chamber via port 22 into a syringe 120. In seven repeat experiments a cell suspension containing between 1-3×10⁷ human foetal lung fibroblast cells type MRC-5 in an initial volume of 1.5 ml of PBS underwent a volume reduction process driven by positive air pressure at the inlet port 20, air being supplied from a syringe connected to inlet fluidic pathway 26, to reach a mean final volume of 300 μl with a standard deviation of 100 μl.

1. Apparatus for filtration of a fret liquid comprising a species in suspension, comprising:
(i) a filter device comprising: a tubular first chamber having a filter provided in a surface thereof, a second chamber in fluid communication with the first chamber through the fitter, the device being provided with an inlet port opening to the first end of the first chamber, an outlet port opening to the second end of the first chamber, and a filtrate port opening to the second chamber, and
(ii) a fluidic pathway from the outlet port,
wherein the apparatus is configured to introduce the first liquid and a second fluid immiscible with the first to the first chamber and the apparatus comprises a flow cut-off means in the fluidic pathway from the outlet port.

2. Apparatus according to claim 1 wherein the flow cut-off means comprises a cut-off valve connected to the outlet port.

3. Apparatus according to claim 1 wherein the flow cut-off means comprises means to prevent fluid flow into a collection device.

4. Apparatus according to any preceding claim wherein the inlet port is configured to receive both the first liquid and the second immiscible fluid.

5. Apparatus according to any preceding claim further comprising a control means configured to set the flow cut-off means in one of a first condition in which flow along the outlet fluidic pathway is prevented and a second condition in which such flow is permitted.

6. Apparatus according to claim 5 wherein the flow cut-off means is a cut-off valve, comprising a control means configured to set the cut-off valve in one of a first position in which the valve is closed and a second position in which the valve is open.

7. Apparatus according to claim 5 or claim 6 wherein the control means is configured to control flow into the first chamber of one or both of the first liquid and the second immiscible fluid.

8. Apparatus according to any of claims 5 to 8 further comprising a suspension inlet fluidic pathway comprising a suspension inlet valve and a second fluid inlet fluidic pathway comprising a second fluid inlet valve, the said fluidic pathways being connected to the first chamber, wherein the control means is configured to control the cut-off valve and one or both of the suspension inlet valve and the second fluid inlet valve.

9. Apparatus according to any preceding claim comprising a reservoir of a second fluid immiscible with the first liquid in fluid communication with a port opening to the first chamber.

10. Apparatus according to any preceding claim wherein the second immiscible fluid is a gas.

11. Apparatus according to any of claims 1 to 9 wherein the second immiscible fluid is a second liquid.

12. Apparatus according to any preceding claim wherein the filter device comprises the cutoff valve.

13. Apparatus according to any preceding claim wherein the cut-off waive is a check valve.

14. Apparatus according to any preceding claim wherein the cutoff valve is in close connection to the first chamber.

15. Apparatus according to any preceding claim further comprising a reservoir of a third liquid miscible with the first liquid in fluid communication with the inlet or the outlet port.

16. Apparatus according to any preceding claim wherein the first chamber has a substantially circular cross section and the filter is disposed in the surface forming the periphery of the first chamber.

17. Apparatus according to any of claims 1 to 15 wherein the filter device comprises one or more substantially planar membranes.

18. Apparatus according to claim 17 wherein the first chamber comprises a base impermeable to the first liquid and the fitter is disposed in the surface of the first chamber distally to the impermeable base.

19. Apparatus according to any preceding claim wherein the filter device comprises a plurality of first chambers.

20. Apparatus according to any preceding claim wherein a first chamber comprises a region having impermeable waits adjacent to the outlet of the chamber.

21. Apparatus according to any preceding claim wherein a wall of the first chamber is inclined downwards towards the outlet port at an angle to the horizontal.

22. Apparatus according to claim 21 wherein the angle is in the range approximately 3 to approximately 85 degrees.

23. Apparatus according to claim 22 wherein the angle is in the range approximately 30 to approximately 80 degrees.

24. Apparatus according to any of claims 1 to 21 wherein the first chamber is arranged vertically.

25. Apparatus according to any preceding claim comprising one or more further ports opening to the first chamber at a position between the inlet pert and the outlet port.

26. Apparatus according to any preceding claim wherein the outlet port of the first chamber is connected to a collection device.

27. A system for processing a liquid containing a species in suspension comprising apparatus according to an preceding claim and a source of liquid containing the species in fluid communication with the inlet of the filter device.

28. A system according to claim 27 further comprising a data source in data communication with the control means operable to control movement of liquid containing the species within the apparatus.

29. A system according to claim 28 wherein the data source comprises a computer and a database external to the apparatus.

30. A system according to claim 26 wherein the data source comprises a data store physically associated with a container housing the liquid containing the species.

31. A method for filtration of a first liquid containing a species in suspension using an apparatus of any one of claims 1 to 26, or a system of any of claims 27 to 30, comprising the steps of:
(i) causing a first liquid containing the species to flow into the first chamber
(ii) introducing a second immiscible fluid into the first chamber while the fluidic pathway from the outlet port is closed, while liquid flows through the filter to the second chamber, to form a concentrated suspension containing the species m the first chamber,
and subsequently
(iii) flowing the concentrated liquid containing the species in suspension formed in step (ii) out from the first chamber.

32. A method according to claim 31 wherein the second fluid is introduced into the first chamber under pressure to force the first liquid through the filter.

33. A method according to claim 31 or 32 wherein the second fluid is a gas.

34. A method according to any of claims 31 to 33 wherein the second fluid is introduced into the first chamber until the volume of first liquid within the first chamber is reduced by between approximately 50% and approximately 98%.

35. A method according to claim 34 wherein the second fluid is introduced into the first chamber until the volume of first liquid within the first chamber is reduced by between approximately 60% and approximately 95%.

36. A method according to any of claims 31 to 35 comprising the further step of separating at least a portion of the second fluid from the first liquid.

37. A method according to any of claims 31 to 36 comprising the further step of mixing a wash liquid with the suspension.

38. A method according to any of claims 31 to 37 wherein the first liquid is a cell suspension comprising a cryoprotectant.

39. A suspension of a species obtained by a method according to any of claims 31 to 38.
